SEQUENCE LISTING (11 sequences)

SEQ ID NO: 1 - protein, 26 aa, synthetic construct
GSLLKVPRKPSPIIFKLKGPKLAVHP

SEQ ID NO: 2 - DNA, 18 bp, synthetic construct
  source 1..18: mol_type = other DNA
ctggccgtggctctcttg

SEQ ID NO: 3 - DNA, 20 bp, synthetic construct
  source 1..20: mol_type = other DNA
ccttggcaaaactgcacctt

SEQ ID NO: 4 - DNA, 20 bp, synthetic construct
  source 1..20: mol_type = other DNA
tgcaataaccacccctgacc

SEQ ID NO: 5 - DNA, 21 bp, synthetic construct
  source 1..21: mol_type = other DNA
tgcgcagaatgagatgagttg

SEQ ID NO: 6 - DNA, 23 bp, synthetic construct
  source 1..23: mol_type = other DNA
atgtggatcagcaagcaggagta

SEQ ID NO: 7 - DNA, 18 bp, synthetic construct
  source 1..18: mol_type = other DNA
gcatttgcggtggacgat

SEQ ID NO: 8 - protein, 38 aa, Homo sapiens
PHVALKPGKLKFIIPSPKRPVKLLSGGNTLHLVSTTKT

SEQ ID NO: 9 - protein, 38 aa, Homo sapiens
PHVALKAGKLKFIIPSPKRPVKLLSGGNTLHLVSTTKT

SEQ ID NO: 10 - protein, 26 aa, Homo sapiens
PHVALKPGKLKFIIPSPKRPVKLLSG

SEQ ID NO: 11 - protein, 26 aa, synthetic construct
  SITE 1: note = D-Glycine
  SITE 2: note = D-Serine
  SITE 3: note = D-Leucine
  SITE 4: note = D-Leucine
  SITE 5: note = D-Lysine
  SITE 6: note = D-Valine
  SITE 7: note = D-Proline
  SITE 8: note = D-Arginine
  SITE 9: note = D-Lysine
  SITE 10: note = D-Proline
  SITE 11: note = D-Serine
  SITE 12: note = D-Proline
  SITE 13: note = D-Isoleucine
  SITE 14: note = D-Isoleucine
  SITE 15: note = D-Phenylalanine
  SITE 16: note = D-Lysine
  SITE 17: note = D-Leucine
  SITE 18: note = D-Lysine
  SITE 19: note = D-Glycine
  SITE 20: note = D-Proline
  SITE 21: note = D-Lysine
  SITE 22: note = D-Leucine
  SITE 23: note = D-Alanine
  SITE 24: note = D-Valine
  SITE 25: note = D-Histidine
  SITE 26: note = D-Proline
GSLLKVPRKPSPIIFKLKGPKLAVHP

(19) United States
(12) Patent Application Publication
(10) Pub. No.: US 2024/0067685 A1
(43) Pub. Date: Feb. 29, 2024

(54) HIDDEN ANTIBIOTICS IN THE HUMAN PROTEOME

(71) Applicant: The Trustees of the University of Pennsylvania, Philadelphia, PA (US)

(72) Inventors: César de la Fuente-Nunez, Philadelphia, PA (US); Angela Arciello, Portici (IT); Angela Cesaro, Philadelphia, PA (US); Marcelo Der Torossian Torres, Philadelphia, PA (US)

(21) Appl. No.: 18/331,331

(22) Filed: Jun. 8, 2023

Related U.S. Application Data
(60) Provisional application No. 63/350,815, filed on Jun. 9, 2022.

(51) Int. Cl. C07K 14/47; A61K 45/06; A61P 31/04

(52) U.S. Cl. CPC C07K 14/4747; A61K 45/06; A61P 31/04; A61K 38/00

(57) ABSTRACT

Provided herein are antimicrobial peptides, as well as methods of treating a microbial infection, methods of treating inflammation, and methods of reducing biofilm formation, using the disclosed antimicrobial peptides.

### CROSS-REFERENCE TO RELATED APPLICATIONS

This application claims the benefit of priority to U.S. Provisional Application No. 63/350,815, filed Jun. 9, 2022, the entire contents of which are incorporated herein by reference.

### GOVERNMENT RIGHTS

This invention was made with government support under R35GM138201 awarded by the National Institutes of Health and HRTRA-21-1-0014 awarded by the Department of Defense. The government has certain rights in the invention.

### SEQUENCE LISTING

The instant application contains a Sequence Listing which has been submitted electronically in XML format and is hereby incorporated by reference in its entirety. Said XML copy, created on Jul. 14, 2023, is named 103241_006945_22- 9974_SL.xml and is 20,183 bytes in size.

### TECHNICAL FIELD

The present disclosure pertains to identification and use of novel antimicrobial agents.

### BACKGROUND

According to the Centers for Disease Control and Prevention (CDC), in 2019, 2.8 million antibiotic-resistant infections occurred in the US, leading to approximately 35,000 deaths¹. Such untreatable infections are projected to reach 10 million people per year worldwide, becoming the leading cause of death in our society². This daunting scenario coincides with the lack of innovation in antibiotic discovery. Most antibiotics available today have been used for over 30 years. These drugs often have unintended side effects, readily select for antibiotic resistance, and, in the face of this resistance, are losing effectiveness³. Thus, there is an urgent need to discover new antimicrobial agents to target drug-resistant infections⁴.

### SUMMARY

Disclosed herein is an antimicrobial peptide, wherein the peptide is SEQ ID NO:1. Also provided are compositions comprising an antimicrobial peptide according to the present disclosure and a pharmaceutically acceptable carrier or excipient.

Also disclosed are methods of treating a microbial infection comprising administering to a subject in need thereof a pharmaceutically effective amount of a disclosed antimicrobial peptide. Also provided are methods of treating inflammation comprising administering to a subject in need thereof a pharmaceutically effective amount of a disclosed antimicrobial peptide. The present methods may further include administering to the subject an additional antimicrobial agent, such as an antibiotic.

The present disclosure also provides methods comprising contacting a biofilm with an effective amount of a disclosed antimicrobial peptide.

Also disclosed are methods for reducing biofilm formation on a surface comprising contacting the surface with an effective amount of a disclosed antimicrobial peptide.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

FIGS. 1A-1D illustrate the antimicrobial activity and synergistic interactions of ApoB-derived encrypted peptides. (FIG. 1A) Schematic representation of the proteolytic release of ApoB-derived encrypted peptides from precursor human plasma apolipoprotein B, the main constituent of low-density lipoproteins; the sequences of each peptide are also reported. FIG. 1A discloses SEQ ID NOS 8-10, respectively, in order of appearance. (FIG. 1B) Antimicrobial activity analyzed by testing peptide concentrations ranging from 0 to 20 μmol L⁻¹ (0-80 μg mL⁻¹ for peptide r(P)ApoB_{L}^{Pro} or r(P)ApoB_{L}^{Ala}, and 0-40 μg mL⁻¹ for peptide r(P)ApoB_{S}^{Pro}) of ApoB-derived encrypted peptides against four bacterial strains; reported data refer to assays performed in triplicate and heat maps show averaged log (CFU mL⁻¹) values. (FIG. 1C) Schematic representation of peptide, colistin and polymyxin B concentrations used in combinatorial therapy. (FIG. 1D) Killing kinetic curves obtained by treating K. pneumoniae ATCC 700603 with the lead combinations of ApoB-derived encrypted peptides and colistin or polymyxin B; curves have been compared with those obtained by incubating cells with single agents at bactericidal concentrations. Note that groups treated with colistin MIC or polymyxin B MIC and colistin 0.127×MIC+r(P)ApoB_{S}^{Pro} 0.093×MIC, polymyxin B 0.25×MIC+r(P)ApoB_{S}^{Pro} 0.125×MIC or polymyxin B 0.33×MIC+r(P)ApoB_{L}^{Pro} 0.112×MIC overlap in the figure.

FIGS. 2A-2E show the antimicrobial effects of ApoB-derived encrypted peptides both alone and in combination, and resistance development studies. Antimicrobial activity of (FIG. 2A) ApoB-derived encrypted peptides and (FIG. 2B) conventional antimicrobial agents against S. epidermidis ATCC 35984, S. aureus ATCC 12600, A. baumannii ATCC 17878, K. pneumoniae ATCC 700603, S. aureus MRSA (WKZ-2), E. faecalis ATCC 29212 and P. aeruginosa wild-type PAO1 strains. Black cells indicate bacterial resistance. The broth microdilution assay was performed using three biological replicates and MIC values were defined as the lowest antimicrobial concentrations needed to reduce the number of bacterial colonies by 100%. (FIG. 2C) Synergistic interactions (expressed as the FIC) of each peptide in combination with conventional antimicrobials against four bacterial strains. In the heat map, synergistic effects are reported in purple, additive effects in light purple and indifferent interactions in white. (FIG. 2D) Morphological analyses by SEM of K. pneumoniae ATCC 700603 strain treated with combinations of r(P)ApoB_{S}^{Pro} and colistin or with the single agents under identical experimental conditions. Red arrows indicate morphological alterations on bacterial membranes caused by peptide treatment. (FIG. 2E) Morphological analyses of wild-type and selected resistant Acinetobacter baumannii ATCC 17878 and Staphylococcus epidermidis ATCC 35984 strains by SEM.

FIGS. 3A-2C depict the mechanism of action and resistance development studies. (FIG. 3A) Schematic representation of ApoB-derived encrypted peptides effects on bacterial transmembrane potential (Δψ). (FIG. 3B) Analysis of fluorescence intensity variation upon bacterial treatment with ApoB-derived encrypted peptides and CATH-2 peptide (positive control) in the presence of DiSC₃(5) dye; data refer to untreated control bacterial cells. Assays have been performed in triplicate and data represent the mean±standard deviation. (FIG. 3C) Evaluation of resistance phenotype development upon prolonged treatment of Acinetobacter baumannii ATCC 17878 and Staphylococcus epidermidis ATCC 35984 with colistin, gentamicin, mupirocin, r(P)ApoB_{L}^{Pro}, r(P)ApoB_{L}^{Ala} and r(P)ApoB_{S}^{Pro}. Assays were performed in duplicate, and data represent the mean fold change in MIC value over time.

FIG. 4 shows the effects of ApoB-derived encrypted peptides on the outer membrane of Gram-negative bacteria. Analysis of the fluorescence intensity variation upon treatment of cells with ApoB-derived peptides and CATH-2 peptide (positive control) in the presence of NPN dye. Assays were performed in triplicate and data represent the mean ±standard deviation.

FIGS. 5A and 5B show the antibiofilm activity of encrypted peptides derived from ApoB. Schematic representation of two stages of bacterial biofilm development. Effects of ApoB-derived encrypted peptides on cells attachment and biofilm formation in the case of (FIG. 5A) A. baumannii ATCC 17878 and (FIG. 5B) S. epidermidis ATCC 35984 by CLSM imaging. Significant differences were indicated as (*P<0.05), (***P<0.001) or (****P<0.0001) for treated versus control samples, each experiment was carried out in triplicate.

FIG. 6 depicts resistance development studies. Evaluation of resistance development in S. aureus ATCC 12600 after prolonged treatment with colistin, gentamicin, mupirocin, r(P)ApoB_{L}^{Pro}, r(P)ApoB_{L}^{Ala}, and r(P)ApoB_{S}^{Pro}. Assays were performed in duplicate and data represent the mean fold change in MIC value over time.

FIGS. 7A-7E show the biocompatibility and anti-inflammatory properties of ApoB-derived encrypted peptides. (FIG. 7A) Schematic representation of the skin barrier. (FIG. 7B) Cytotoxic effects of increasing concentrations of r(P)ApoB_{L}^{Pro} (blue), r(P)ApoB_{S}^{Pro} (orange) and r(P)ApoB_{L}^{Ala} (pink) on HDF (human dermal fibroblasts) and A431 (human epidermoid carcinoma cells) cell lines over time. (FIG. 7C) LDH release upon treatment of HDF and A431 cells with ApoB-derived encrypted peptides. The positive control was obtained by treating cells with lysis buffer. (FIG. 7D) ApoB-derived encrypted peptides effects on IL-8 and IL-6 expression in HDF cells infected with S. aureus ATCC 12600 by RT-qPCR. (FIG. 7E) Effects of ApoB-derived encrypted peptides on HDAC activity in HaCaT cells. HDAC activity is expressed as a percentage of the activity determined in untreated control cells. Significant differences were indicated as (*P<0.05) for treated versus control samples.

FIGS. 8A and 8B depict the synergistic interactions between ApoB-derived encrypted peptides in (FIG. 8A) 2- and (FIG. 8B) 3-way combinations. Synergistic interactions (expressed as FIC indexes) of two or three peptides when used in combination therapy against each target bacterium. Synergistic, additive, and indifferent interactions are displayed in purple, light purple, and white, respectively.

FIGS. 9A-9E show the stability, antimicrobial activity, and cytotoxic profile of retro-inverso synthetic peptide. (FIG. 9A) Schematic representation of retro-inverso peptide design leading to (ri)-r(P)ApoB_{S}^{Pro}. FIG. 9A discloses SEQ ID NOS 10, 1, and 11, respectively, in order of appearance. (FIG. 9B) Antimicrobial activity of (ri)-r(P)ApoB_{S}^{Pro} (μmmol L⁻¹) against four bacterial strains; reported data refer to assays performed in triplicate and heat maps show averaged log (CFU mL⁻¹) values. (FIG. 9C) Antibacterial activity of (ri)-r(P)ApoB_{S}^{Pro} peptide against the same four bacterial strains after preincubation in 10% serum. Reported data refer to assays performed in triplicate and the fold changes in antimicrobial activity are calculated as ratio between peptide MIC values obtained after and before incubation for 1 h and 16 h in the presence of 10% serum at 37° C. (statistical significance was determined using two-way ANOVA followed by Dunnett's test, **p<0.01 and ****p<0.0001). (FIG. 9D) Resistance to degradation of (ri)-r(P)ApoBs^{Pro} exposed to fetal bovine serum (FBS) proteases for 4 h. (FIG. 9E) Cytotoxic effects of increasing concentrations of (ri)-r(P)ApoBs^{Pro} on HDF (human dermal fibroblasts) cells. No significant differences were observed for the time points assessed.

FIG. 10 depicts the antibiofilm activity of ApoB-derived encrypted peptides. Anti-biofilm activity of ApoB-derived encrypted peptides on S. epidermidis ATCC 35984, S. aureus ATCC 12600, A. baumannii ATCC 17878 and K. pneumoniae ATCC 700603 strains. The effects of increasing concentrations of r(P)ApoB_{L}^{Pro} (blue), r(P)ApoB_{L}^{Ala} (pink), and r(P)ApoB_{S}^{Pro} (orange) peptides were evaluated on cells attachment, biofilm formation, or on preformed biofilm. Biofilm was stained with crystal violet and measured at 600 nm. Data represent the mean (±standard deviation, SD) of at least three independent experiments, each one carried out with triplicate determinations. Significant differences were indicated as *(P<0.05), **(P<0.01), ***(P<0.001) for treated versus control samples.

FIGS. 11A-11D depict the In vivo activity of natural and synthetic encrypted peptides derived from human ApoB. (FIG. 11A) Schematic representation of the in vivo experimental design. The back of mice was shaved, and an abrasion was generated to damage the stratum corneum and the upper layer of the epidermis. Subsequently, an aliquot of 50 μL containing 10⁶ CFU of P. aeruginosa PAO1 in PBS was inoculated over each defined area. One hour after the infection, peptides at 20 or 200 μmol L⁻¹ were administered to the infected area. Four animals per group were euthanized at day 2 or 4 post-infection and the area of scarified skin was excised and homogenized for 20 min (25 Hz). (FIG. 11B) Homogenized samples were serially diluted for CFU quantification (statistical significance was determined using two-way ANOVA followed by Dunnett's test, ***p<0.001). (FIG. 11C) Mouse body weight measurements were performed throughout the experiment and normalized by the body weight at the beginning of the experiment. (FIG. 11D) In vivo dose-response curve obtained by administering increasing concentrations of each peptide at the infection site one hour after the infection was established(statistical significance was determined using two-way ANOVA followed by Dunnett's test, ***p<0.001).

FIG. 12 shows the activity of ApoB-derived encrypted peptides on A. baumannii biofilm formation. Effects of ApoB-derived encrypted peptides on A. baumannii ATCC17878 biofilm formation.

FIG. 13 illustrates the biocompatibility of ApoB-derived encrypted peptides. Cytotoxic effects of increasing concentrations (0-25-50-100-200 μmol L⁻¹) of r(P)ApoB_{L}^{Pro} (blue), r(P)ApoB_{L}^{Ala} (pink), and r(P)ApoB_{S}^{Pro} (orange) on HDF (human dermal fibroblasts) and A431 (human epidermoid carcinoma cells) cell lines over time (24, 48 and 72 h). Assays were performed in triplicate and data represent the average cell viability obtained comparing each sample with the untreated cells (control) in each case.

FIG. 14 shows the anti-inflammatory properties of ApoB-derived encrypted peptides. Effects of ApoB-derived encrypted peptides at two different concentrations (5 or 20 μmol L⁻¹) on IL-8 and IL-6 expression in HDF cells infected with A. baumannii ATCC 17878 by RT-qPCR. Assays were performed in duplicate, and each sample was treated with peptide (r(P)ApoB_{L}^{Pro}, r(P)ApoB_{L}^{Ala}, r(P)ApoB_{S}^{Pro}, or CATH-2) and compared with the untreated control. Significant differences were indicated as *(P<0.05) and **(P<0.01) for treated versus untreated samples.

FIG. 15 depicts the effect of human dermal fibroblasts stimulated with LPS and LTA. IL-8 and IL-6 expression as determined by RT-qPCR. HDF cells were either unstimulated (negative control) or stimulated for 4 h with 1 μg mL⁻¹ of LPS from E. coli or LTA from S. aureus.

FIG. 16 shows the anti-inflammatory properties of ApoB-derived encrypted peptides. Effects of ApoB-derived encrypted peptides at two different concentrations (5 or 20 μmol L⁻¹) on IL-8 and IL-6 expression in HDF cells stimulated with LPS from E. coli by RT-qPCR. Assays were performed in duplicate and each sample treated with peptide (r(P)ApoB_{L}^{Pro}, r(P)ApoB_{L}^{Ala}, r(P)ApoB_{S}^{Pro} or CATH-2) was compared with the untreated control. Significant differences between the groups were indicated as *(P<0.05), **(P<0.01) and ***(P<0.001) for treated versus the untreated sample.

FIG. 17 shows the immunomodulatory effects of encrypted peptides. Induction of IL-8, TNFα and MCP-1 on THP-1 cells treated with ApoB-derived encrypted peptides with respect to the control group containing LPS from P. aeruginosa PAO1.

FIG. 18 shows the antimicrobial effects of (ri)-r(P)ApoB_{S}^{Pro} peptide. Antimicrobial activity of (ri)-r(P)ApoB_{S}^{Pro} against S. epidermidis ATCC 35984, S. aureus ATCC 12600, A. baumannii ATCC 17878, K. pneumoniae ATCC 700603, S. aureus MRSA (WKZ-2), E. faecalis ATCC 29212, and P. aeruginosa wild-type PAO1.

FIG. 19 illustrates the biocompatibility of (ri)-r(P)ApoB_{S}^{Pro} peptide. Cytotoxic effects of increasing concentrations (0, 25, 50, 100 and 200 μmol L⁻¹) of (ri)-r(P)ApoB_{S}^{Pro} on HDF (human dermal fibroblasts) cell lines over time (24-48-72 h). Assays have been performed in triplicate and data represent the average of the cell viability obtained comparing each sample with the untreated cells (control).

FIG. 20 depicts the results of a study involving monitoring mouse body weight over the course of the experiment as a proxy for toxicity. The measurements were performed 2- and 4-days post-infection and post-peptide treatment at different doses (50, 100, and 150 μmol L⁻¹), and normalized by the body weight at the beginning of the experiment. Changes in weight lower than 20% were not considered a sign of toxicity for treated and untreated controls. Four animals were used per group.

### DETAILED DESCRIPTION OF ILLUSTRATIVE EMBODIMENTS

The presently disclosed inventive subject matter may be understood more readily by reference to the following detailed description taken in connection with the accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a treatment” is a reference to one or more of such treatments and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.” The phrase “at least about x” is intended to embrace both “about x” and “at least x”. It is also understood that where a parameter range is provided, all integers within that range, and tenths thereof, are also provided by the invention. For example, “2-5 hours” includes 2 hours, 2.1 hours, 2.2 hours, 2.3 hours etc., up to 5 hours.

Publications with potential relevance to the presently disclosed subject matter are cited in the present disclosure using superscripted numerals that correspond to the numbered references that are listed in the present disclosure under the heading “References”, infra.

Encrypted peptides have been recently found in the human proteome and represent a potential class of antibiotics. Disclosed herein are peptides derived from the human apolipoprotein B (residues 887-922) that exhibit potent antimicrobial activity, including against drug-resistant Klebsiella pneumoniae, Acinetobacter baumannii, and Staphylococci both in vitro and in an animal model. The peptides have excellent cytotoxicity profiles, target bacteria by depolarizing and permeabilizing their cytoplasmic membrane, inhibit biofilms, and display anti-inflammatory properties. Importantly, the peptides potentiate the activity of conventional antibiotics against bacteria and do not select for bacterial resistance. These results provide a link between human plasma and innate immunity and point to the blood as a source of much-needed antimicrobials.

Provided herein are peptides of SEQ ID NO:1: GSLLKVPRKPSPIIFKLKGPKLAVHP.

Also disclosed are peptides that are at least 90% homologous to SEQ ID NO:1. In some embodiments, the inventive peptides are at least 95% homologous to SEQ ID NO:1. In certain embodiments, the inventive peptides have amino acid sequences that respectively vary from SEQ ID NO:1 by no more than three individual amino acids. For example, the inventive peptide may have an amino acid sequence that varies from SEQ ID NO:1 by three, two, or one individual amino acids.

Also provided herein are compositions comprising a peptide according to any of the embodiments disclosed herein, and a pharmaceutically acceptable carrier, diluent, or excipient. The present disclosure also provides methods of treating a microbial infection comprising administering to a subject in need thereof a pharmaceutically effective amount of a peptide according to any of the disclosed embodiments. The present disclosure also provides methods of treating inflammation in a subject comprising administering to a subject in need thereof a pharmaceutically effective amount of a peptide according to any of the disclosed embodiments. As described above, the present inventors have discovered that the peptides disclosed herein possess antimicrobial characteristics, and therefore represent alternatives both to traditional antibiotic compounds to which microbial resistance has arisen or is likely to arise, and to naturally occurring compounds that possess unacceptably high levels of toxicity to mammalian cells. The present inventors have also discovered that the inventive peptides have anti-inflammatory properties, such as by reducing the expression of one or more inflammatory markers, such as IL-6 or IL-8.

As used herein, the phrase “therapeutically effective amount” refers to the amount of active agent (here, the antimicrobial peptide) that elicits the biological or medicinal response that is being sought in a tissue, system, animal, individual or human by a researcher, veterinarian, medical doctor or other clinician, which includes one or more of the following:
(1) at least partially preventing the disease or condition or a symptom thereof; for example, preventing a disease, condition or disorder in an individual who may be predisposed to the disease, condition or disorder but does not yet experience or display the pathology or symptomatology of the disease;
(2) inhibiting the disease or condition; for example, inhibiting a disease, condition or disorder in an individual who is experiencing or displaying the pathology or symptomatology of the disease, condition or disorder (i.e., including arresting further development of the pathology and/or symptomatology); and
(3) at least partially ameliorating the disease or condition; for example, ameliorating a disease, condition or disorder in an individual who is experiencing or displaying the pathology or symptomatology of the disease, condition or disorder (i.e., including reversing the pathology and/or symptomatology).

The antimicrobial peptides according to the present disclosure may be provided in a composition that is formulated for any type of administration. For example, the compositions may be formulated for administration orally, topically, parenterally, enterally, or by inhalation (e.g., intranasally). The active agent may be formulated for neat administration, or in combination with conventional pharmaceutical carriers, diluents, or excipients, which may be liquid or solid. The applicable solid carrier, diluent, or excipient may function as, among other things, a binder, disintegrant, filler, lubricant, glidant, compression aid, processing aid, color, sweetener, preservative, suspensing/dispersing agent, tablet-disintegrating agent, encapsulating material, film former or coating, flavoring agent, or printing ink. Any material used in preparing any dosage unit form is preferably pharmaceutically pure and substantially non-toxic in the amounts employed. In addition, the active agent may be incorporated into sustained-release preparations and formulations. Administration in this respect includes administration by, inter alia, the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol, and rectal systemic.

In powders, the carrier, diluent, or excipient may be a finely divided solid that is in admixture with the finely divided active ingredient. In tablets, the active ingredient is mixed with a carrier, diluent or excipient having the necessary compression properties in suitable proportions and compacted in the shape and size desired. For oral therapeutic administration, the active compound may be incorporated with the carrier, diluent, or excipient and used in the form of ingestible tablets, buccal tablets, troches, capsules, elixirs, suspensions, syrups, wafers, and the like. The amount of active agent(s) in such therapeutically useful compositions is preferably such that a suitable dosage will be obtained.

Liquid carriers, diluents, or excipients may be used in preparing solutions, suspensions, emulsions, syrups, elixirs, and the like. The active ingredient of this invention can be dissolved or suspended in a pharmaceutically acceptable liquid such as water, an organic solvent, a mixture of both, or pharmaceutically acceptable oils or fat. The liquid carrier, excipient, or diluent can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators.

Suitable solid carriers, diluents, and excipients may include, for example, calcium phosphate, silicon dioxide, magnesium stearate, talc, sugars, lactose, dextrin, starch, gelatin, cellulose, methyl cellulose, ethylcellulose, sodium carboxymethyl cellulose, microcrystalline cellulose, polyvinylpyrrolidine, low melting waxes, ion exchange resins, croscarmellose carbon, acacia, pregelatinized starch, crospovidone, HPMC, povidone, titanium dioxide, polycrystalline cellulose, aluminum methahydroxide, agar-agar, tragacanth, or mixtures thereof.

Suitable examples of liquid carriers, diluents and excipients, for example, for oral, topical, or parenteral administration, include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil), or mixtures thereof.

For parenteral administration, the carrier, diluent, or excipient can also be an oily ester such as ethyl oleate and isopropyl myristate. Also contemplated are sterile liquid carriers, diluents, or excipients, which are used in sterile liquid form compositions for parenteral administration. Solutions of the active agents can be prepared in water suitably mixed with a surfactant, such as hydroxypropylcellulose. A dispersion can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier, diluent, or excipient may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In some instances, the antimicrobial peptides themselves may be sufficient to prevent contamination by microorganisms. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active agent in the pharmaceutically appropriate amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and freeze drying techniques that yield a powder of the active ingredient or ingredients, plus any additional desired ingredient from the previously sterile-filtered solution thereof.

Thus, an antimicrobial peptide may be in the present compositions and methods in an effective amount by any of the conventional techniques well-established in the medical field. For example, the administration may be in the amount of about 0.1 mg/day to about 500 mg per day. In some embodiments, the administration may be in the amount of about 250 mg/kg/day. Thus, administration may be in the amount of about 0.1 mg/day, about 0.5 mg/day, about 1.0 mg/day, about 5 mg/day, about 10 mg/day, about 20 mg/day, about 50 mg/day, about 100 mg/day, about 200 mg/day, about 250 mg/day, about 300 mg/day, or about 500 mg/day.

The microbial infection that may be treated according to the present methods may be, for example, viral or bacterial. When the microbial infection is viral, it may include any known viral pathogen. When the microbial infection is bacterial, it may include any known bacterial pathogen. The bacterial strain may be Gram-negative or Gram-positive. In certain embodiments, the infection may include Escherichia coli ATCC11775, Pseudomonas aeruginosa PAO1, Pseudomonas aeruginosa PA14, Staphylococcus aureus ATCC12600, Staphylococcus epidermidis ATCC 35984, Staphylococcus aureus MRSA (WK7-2), Enterococcus faecalis ATCC 29212, Escherichia coli AIC221, E. coli AIC222, Klebsiella pneumoniae ATCC133883, and Acinetobacter baumannii ATCC19606, or any combination thereof.

It has been discovered that the presently disclosed antimicrobial peptides can potentiate the activity of conventional antimicrobial agents, such as antibiotics, in an additive or synergistic manner. Accordingly, the present methods may further include administering to the subject an additional antimicrobial agent (i.e., an antimicrobial agent other than the peptide itself), and the present compositions can further comprise an additional antimicrobial agent. In preferred embodiments, the additional antimicrobial agent is an antibiotic. In some embodiments, the additional antimicrobial agent is active against bacterial membranes, is active against bacterial cell walls, is an inhibitor of bacterial protein synthesis, or is any combination of these.

Also disclosed are methods comprising contacting a biofilm with an effective amount of an antimicrobial peptide according to the present disclosure. Such methods may be effective to remove or reduce the presence of an unwanted biofilm, such as in hospitals or other medical (e.g., surgical) settings, in sewer and filtration systems, in industrial settings, on equipment involved in food preparation or manufacture, in aquaculture or hydroponics, or in any other context that is prone to unwanted biofilm formation.

In accordance with the methods of treating a microbial infection in a subject or the methods comprising contacting a biofilm according to the present disclosure, microbes against which the present antimicrobial peptides are effective may be, for example, any unicellular organism, such as gram-negative bacteria, gram-positive bacteria, protozoa, viruses, bacteriophages, and archaea. The present peptides can have an antimicrobial effect with respect to any such microbe. Examples of bacteria against which the present compounds are effective to cause reduction in numbers include gram positive bacteria and gram negative bacteria, for example, Salmonella enterica, Listeria monocytogenes, Escherichia coli, Clostridium botulinum, Clostridium difficile, Campylobacter, Bacillus cereus, Vibrio parahaemolyticus, Vibrio cholerae, Vibrio vulnificus, Staphylococcus aureus, Yersinia enterocolitica, Shigella, Moraxella spp., Helicobacter, Stenotrophomonas, Bdellovibrio, L egionella spp. (e.g., pneumophila), Neisseria gonorrhoeae, Neisseria meningitidis, Haemophilus influenzae, Acinetobacter baumannii, Klebsiella pneumoniae, Pseudomonas aeruginosa, Proteus mirabilis, Enterobacter cloacae, Serratia mar cescens, Helicobacter pylori, Salmonella enteritidis, Salmonella typhi, and combinations thereof. Examples of Salmonella enterica serovars that can be reduced using the compounds of the disclosure include, for example, Salmonella enteriditis, Salmonella typhimurium, Salmonella poona, Salmonella heidelberg, and Salmonella anatum. In certain embodiments, the biofilm may comprise Escherichia coli ATCC11775, Pseudomonas aeruginosa PAO1, Pseudomonas aeruginosa PA14, Staphylococcus aureus ATCC12600, Staphylococcus epidermidis ATCC 35984, Staphylococcus aureus MRSA (WK7-2), Enterococcus faecalis ATCC 29212, Escherichia coli AIC221, E. coli AIC222, Klebsiella pneumoniae ATCC133883, Acinetobacter baumannii ATCC19606, or any combination thereof. Exemplary viruses against which the present peptides are effective to cause reduction in numbers include coronaviruses, rhinoviruses, and influenza viruses.

### EXAMPLES

The present invention is further defined in the following Examples. It should be understood that these examples, while indicating preferred embodiments of the invention, are given by way of illustration only, and should not be construed as limiting the appended claims. From the above discussion and these examples, one skilled in the art can ascertain the essential characteristics of this invention, and without departing from the spirit and scope thereof, can make various changes and modifications of the invention to adapt it to various usages and conditions.

### Example 1—Antimicrobial Properties of ApoB-derived Encrypted Peptides

Numerous precursor proteins containing encrypted peptides with biological functions that are unrelated to those of the parent protein have been recently found throughout the human body, thus representing an alternative source for antibiotic discovery.6,9 Plasma lipoproteins or apolipoproteins, such as human apolipoprotein B (ApoB-100), are water-soluble complexes composed of lipids and one or more proteins.¹⁵ The concentration of ApoB in normal plasma is approximately 1.1 mg mL⁻¹. ¹⁶ Plasma ApoB, in addition to its physiological role, seems to play an important role in bacterial neutralization.¹⁷ Using an algorithmic approach selecting for physicochemical features as a scoring function, ¹⁸ encrypted peptides within the sequence of human ApoB (amino acids 887-922) were identified. A promising antimicrobial region was identified according to the computational scores assigned to the amino acid sequences of two ApoB-100 isoforms. Next, produced recombinantly in Escherichia coli were three versions of the identified encrypted peptide, namely r(P)ApoB_{L}^{Pro}, r(P)ApoB_{S}^{Pro} and r(P)ApoB_{L}^{Ala}. These sequences present a Pro residue at the N-terminal extremity because of the acidic cleavage of an Asp-Pro bond necessary to excise peptides of interest from the rest of the recombinant construct. ^{5,7,13,19} The ApoB peptide variants were labeled with Pro and Ala indicating their amino acid residue in position 7, which corresponds to the mutation differentiating the two isoforms. The labels L and S refer to a longer or a shorter version of the identified amino acid sequence and correspond to the relative and absolute scores, respectively, generated by the algorithm. ⁵

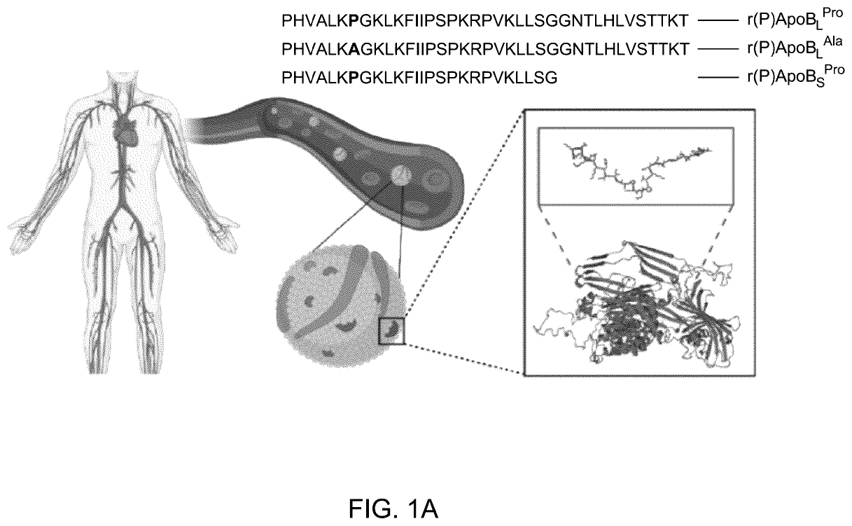

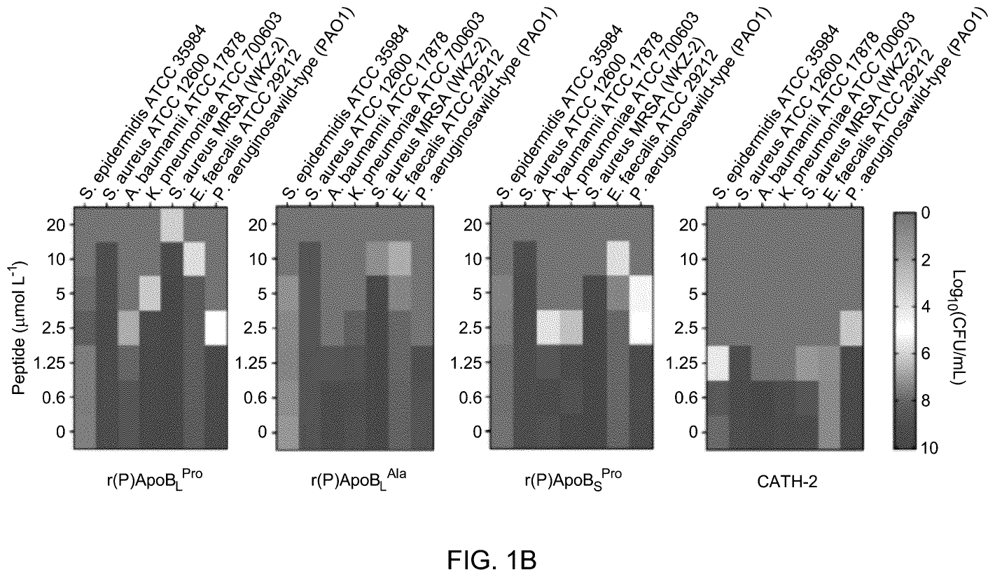

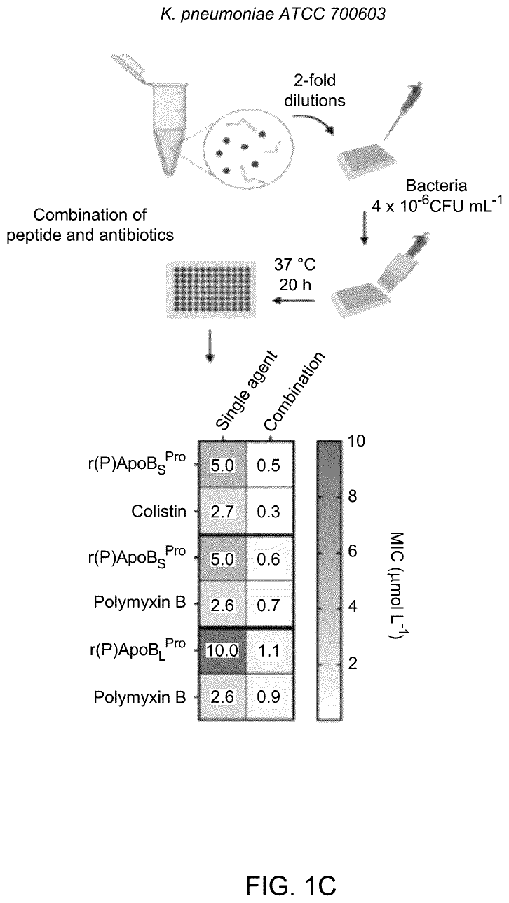

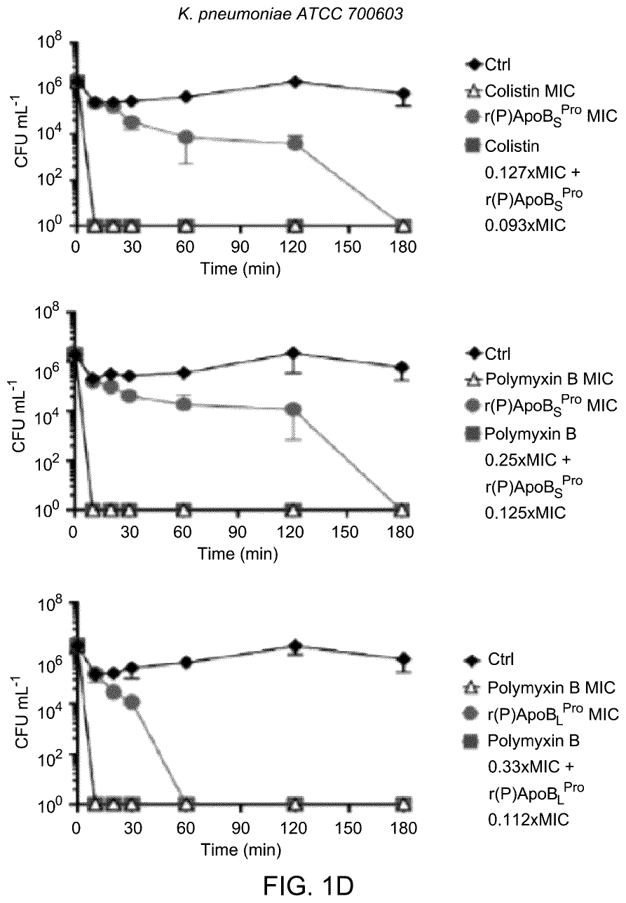

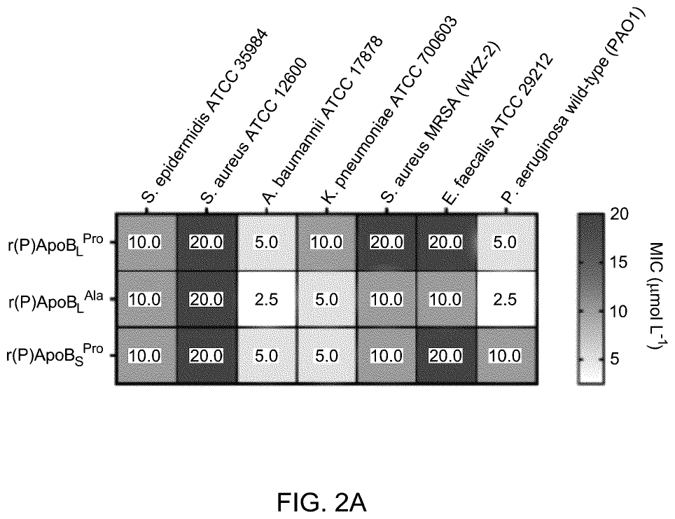

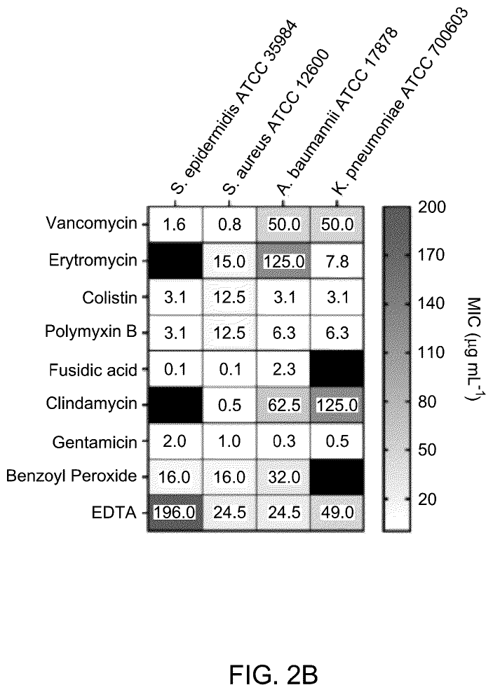

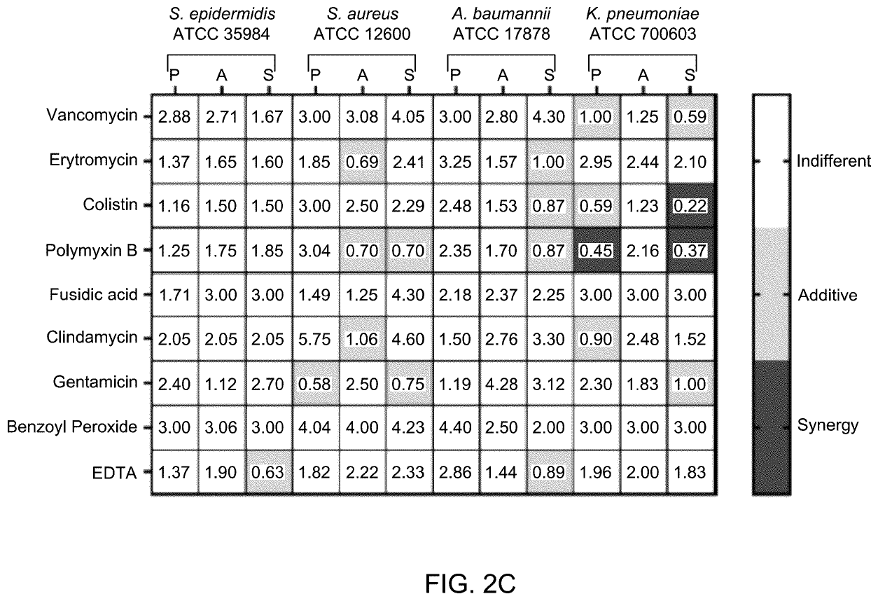

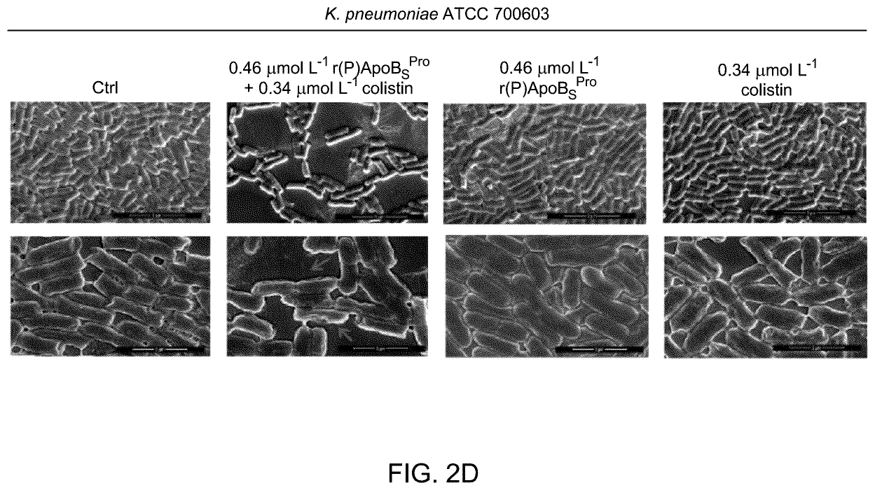

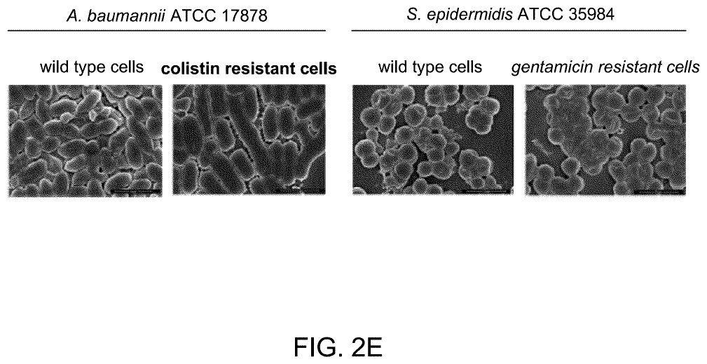

First, the antimicrobial activity of ApoB-derived encrypted peptides was assessed against the following bacterial pathogens: Staphylococcus epidermidis ATCC 35984, Staphylococcus aureus ATCC 12600, Acinetobacter baumannii ATCC 17878, Klebsiella pneumoniae ATCC 700603, Staphylococcus aureus MRSA (WK7-2), Enterococcus faecalis ATCC 29212, and Pseudomonas aeruginosa wild-type (PAO1) using broth microdilution assays 20 to determine their minimal inhibitory concentration (MIC) values, experimentally defined as the lowest antimicrobial concentrations that entirely inhibit bacterial growth. The peptides were found to exert significant antibacterial effects (MICs ranging from 2.5-20 μmol L⁻¹) against all the bacterial strains tested. Staphylococcus epidermidis ATCC 35984, Staphylococcus aureus ATCC 12600, Staphylococcus aureus MRSA (WK7-2), and Enterococcus faecalis ATCC 29212 were found to be susceptible to the encrypted peptides at 10-20 μmol L⁻¹. The peptides were even more active against Acinetobacter baumannii ATCC 17878, Klebsiella pneumoniae ATCC 700603, and Pseudomonas aeruginosa wild-type (PAO1) with MIC values ranging from 2.5 to 5 μmol L⁻, thus indicating the increased ability of the peptides to target Gram-negative bacteria (FIGS. 1B, 2A and Table S1). The antimicrobial activity of these peptides (2.5-20 μmol L⁻¹) is similar to that of another peptide identified in human apolipoprotein E¹² and other potent peptide antimicrobials such as TsAP-2, HM2 & HM5, ranalexin and stylisin 2. ²¹

TABLE S1
MIC values (μmol L⁻¹) of ApoB encrypted peptides.
Antimicrobial activity of r(P)ApoB_{L}^{Pro}, r(P)ApoB_{L}^{Ala} and
r(P)ApoB_{S}^{Pro} against S. epidermidis ATCC 35984, S. aureus ATCC
12600, A. baumannii ATCC 17878, K. pneumoniae ATCC 700603,
S. aureus MRSA (WKZ-2), E. faecalis ATCC 29212 and
P. aeruginosa wild-type PAO1 strains.

| | MIC (μmol L⁻¹) | | |
|---|---|---|---|
| | r(P)ApoB_{L}^{Pro} | r(P)ApoB_{L}^{Ala} | r(P)ApoB_{S}^{Pro} |
| S. epidermidis | 10 | 10 | 10 |
| ATCC 35984 | | | |
| S. aureus | 20 | 20 | 20 |
| ATCC 12600 | | | |
| A. baumannii | 5 | 2.5 | 5 |
| ATCC 17878 | | | |
| K. pneumoniae | 10 | 5 | 5 |
| ATCC 700603 | | | |
| S. aureus | 20 | 10 | 10 |
| MRSA (WKZ-2) | | | |
| E. faecalis | 20 | 10 | 20 |
| ATCC 29212 | | | |
| P. aeruginosa | 5 | 2.5 | 10 |
| wild type (PAO1) | | | |

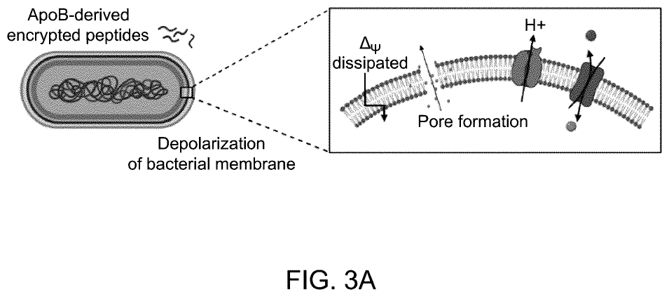

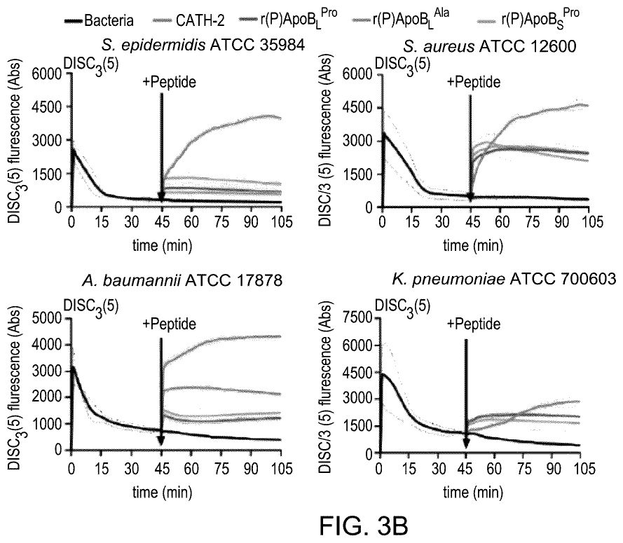

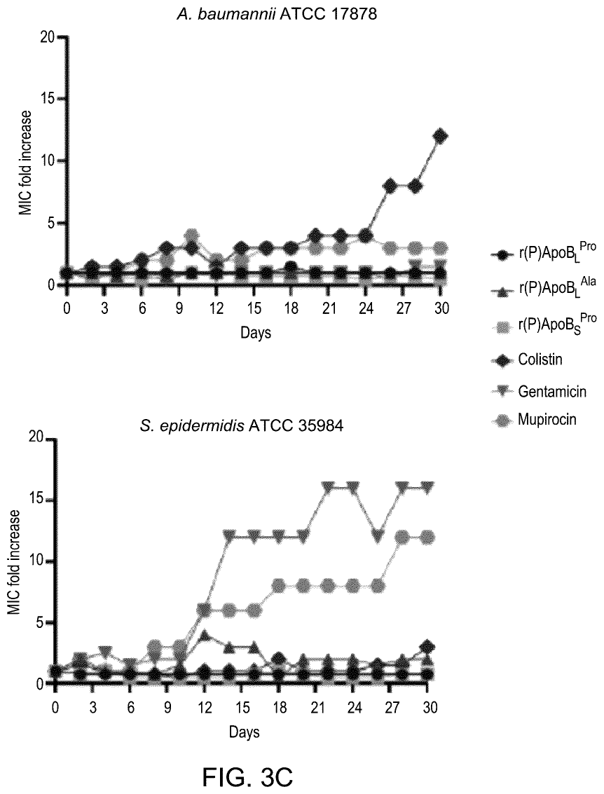

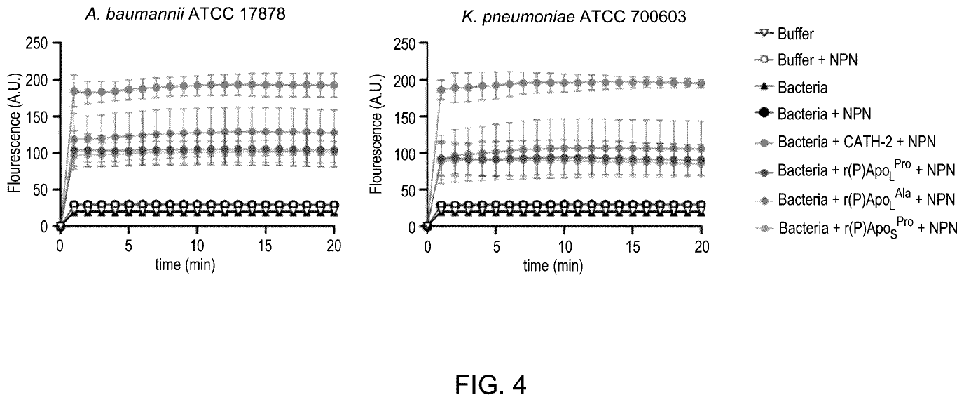

To understand the antibacterial mechanism of action of ApoB-derived peptides, their ability to depolarize bacterial membranes was evaluated by using the voltage-sensitive dye DiSC₃(5) in the presence of live bacterial cells. DiSC₃(5) is a cationic membrane-permeable fluorescent dye that penetrates lipid bilayers and accumulates in polarized cells. ²² The aggregation of DiSC₃(5) molecules lead to a fluorescence quenching effect, lasting about 45 min in our experiments. Upon membrane depolarization, the dye molecules are rapidly released because of their cationic nature, leading to increased fluorescence intensity. ²³ Peptide CATH-2 was used as a positive control since it is known to exert antimicrobial activity by depolarizing the cytoplasmic membrane of bacteria. ^{24,25} In these experiments, an increase in fluorescence intensity, indicating membrane depolarization, was observed in all pathogens tested when treated with ApoB-derived peptides in the presence of the voltage-sensitive dye DiSC₃(5) (FIGS. 3A and 3B). To provide more insights into the mechanisms of action of ApoB-derived peptides, outer membrane permeabilization assays were performed using the Gram-negative bacteria A. baumannii ATCC 17878 and K. pneumoniae ATCC 700603 (FIG. 4). All peptides were found to permeabilize the outer membrane as indicated by an increase in the fluorescent signal associated with the lipophilic dye NPN. The fluorescent NPN probe produced a weak fluorescence in aqueous environments (control samples without peptides), but its fluorescence increased upon contact with the lipidic environment generated by bacterial membrane damage induced by peptide treatment (FIG. 4). These data clearly reveal the ability of ApoB-derived peptides to damage and permeabilize the bacterial outer membrane of Gram-negative bacteria. Hence, altogether, these data demonstrate that the peptides induced membrane depolarization and outer membrane permeabilization, thus leading to variations in the membrane potential, membrane damage, and eventual cell death, mechanisms that are shared with other peptides including several cathelicidins, melittin and the ion channel-forming gramicidin D. ²⁶⁻³⁰

Next, the ability of the peptides to potentiate the antimicrobial activity of conventional antibiotics via synergistic interactions was assessed. Indeed, through synergistic or additive interactions, antimicrobial agents can significantly reduce their therapeutic dose, thus minimizing undesired side effects such as the selection of bacterial resistant phenotypes. ^{31,32} To assess such interactions, checkerboard assays were performed, and the Fractional Inhibitory Concentration (FIC) index was determined in each case. ³³⁻³⁵ First, the antimicrobial activity of each peptide was determined when used as a monotherapy (FIG. 1B and Table S2).

TABLE S2
MIC values (μmol L⁻¹) of conventional antimicrobial agents.
Antimicrobial activity of vancomycin, erythromycin, colistin, polymyxin
B, fusidic acid, clindamycin, gentamicin, benzoyl peroxide and EDTA against
S. epidermidis ATCC 35984, S. aureus ATCC 12600, A. baumannii
ATCC 17878 and K. pneumoniae ATCC 700603 strains.

| | MIC values (μg mL⁻¹) | | | |
|---|---|---|---|---|
| | Staphylococcus | Staphylococcus | Acinetobacter | Klebsiella |
| | epidermidis | aureus | baumannii | pneumoniae |
| Antibiotics | ATCC 35984 | ATCC 12600 | ATCC 17878 | ATCC 700603 |
| Vancomycin | 1.56 | 0.78 | 50 | 50 |
| Erytromycin | Resistant | 15 | 125 | 7.8 |
| Colistin | 3.12 | 12.5 | 3.12 | 3.12 |
| Polymyxin B | 3.12 | 12.5 | 6.25 | 6.25 |
| Fusidic acid | 0.06 | 0.06 | 2.25 | Resistant |
| Clindamycin | Resistant | 0.5 | 62.5 | 125 |
| Gentamicin | 2 | 1 | 0.25 | 0.5 |
| Benzoyl Peroxide | 16 | 16 | 32 | Resistant |
| EDTA | 196 | 24.5 | 24.5 | 49 |

Checkerboard assays revealed widespread additive effects (0.5>FIC index>1; light purple color in FIG. 2C) when peptides were combined with conventional antimicrobials, such as vancomycin, erythromycin, gentamicin, clindamycin and EDTA. Importantly, peptides r(P)ApoB_{L}^{Pro} and r(P)ApoB_{S}^{Pro} synergized with the LPS binders polymyxin B and colistin against the Gram-negative pathogen Klebsiella pneumoniae ATCC 700603 (FIG. 1C; FIC index≤0.5; purple signals in FIG. 2C), thus significantly reducing both the peptide and antibiotic doses needed to eradicate this bacterium. Altogether, synergistic effects were detected when testing ApoB-derived encrypted peptides in combination with antibiotics that are active toward bacterial membranes (e.g., polymyxin B and colistin), whereas additive interactions were observed when combined with antibiotics that inhibit protein synthesis (e.g., erythromycin, clindamycin, and gentamicin) and target the bacterial cell wall (e.g., vancomycin). The synergistic effects observed between the encrypted peptides and conventional antibiotics were further confirmed by changes in cell morphology observed in scanning electron microscopy (SEM) assays (FIG. 2D). Combinations of r(P)ApoB_{S}^{Pro} (0.093×MIC=0.46 μmol L⁻¹) and colistin (0.127×MIC=0.34 μmol L⁻¹) altered the morphology of Klebsiella pneumoniae ATCC 700603 and substantially decreased bacterial viability (FIG. 2D) at non-antimicrobial concentrations for each agent when tested alone (FIG. 2D).

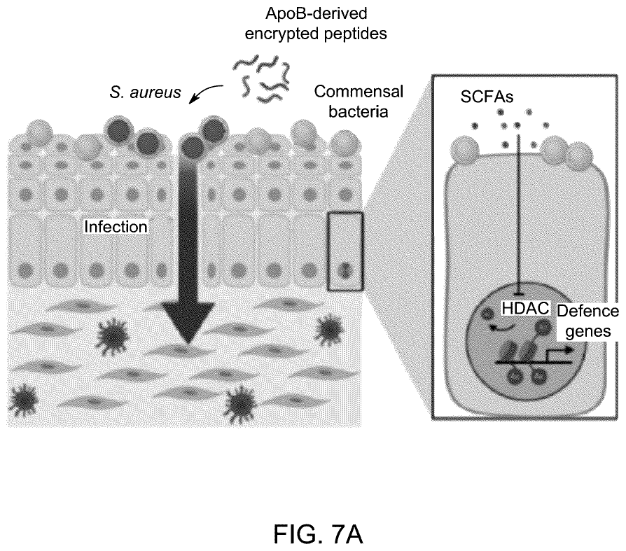

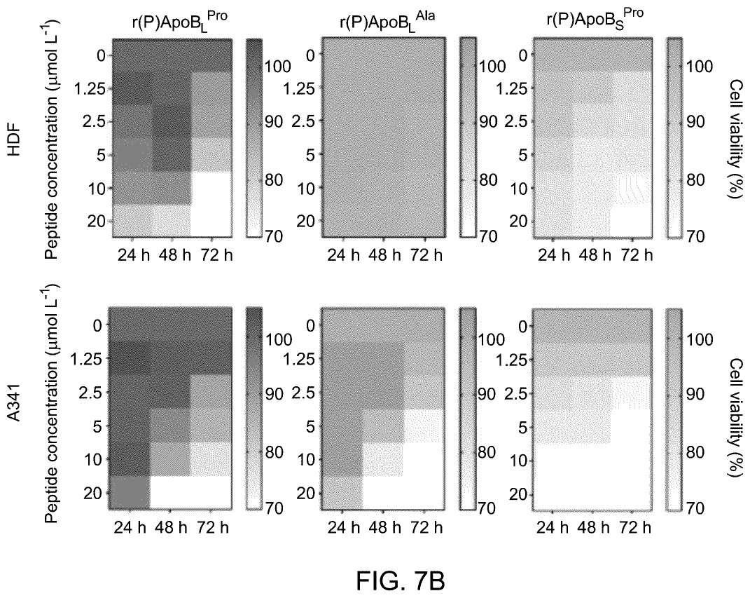

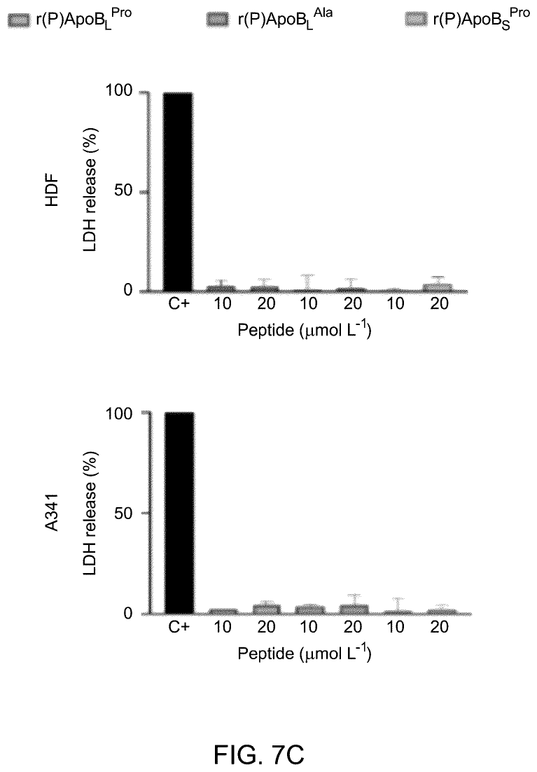

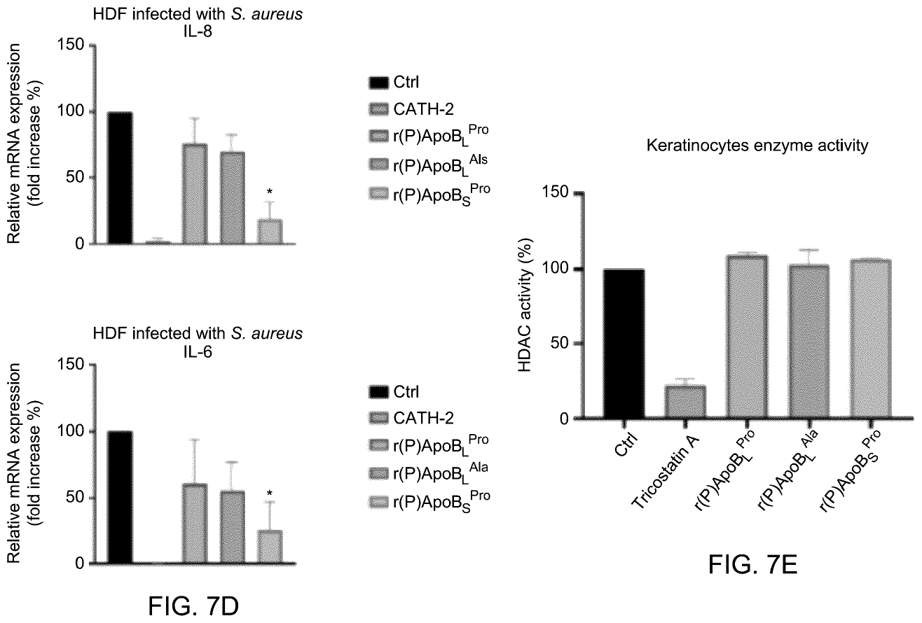

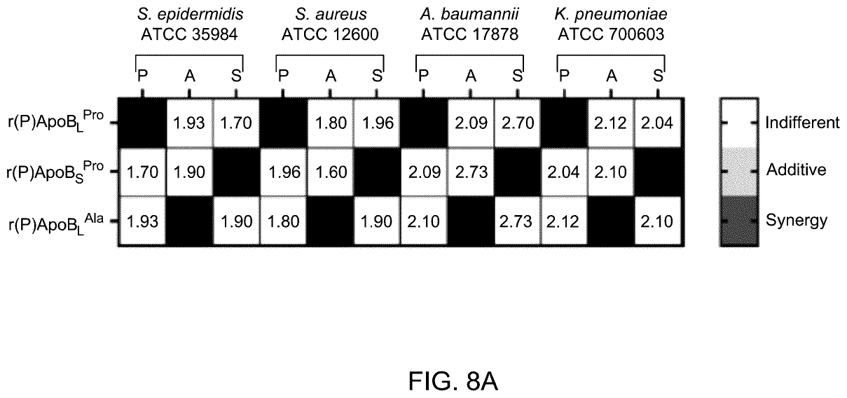

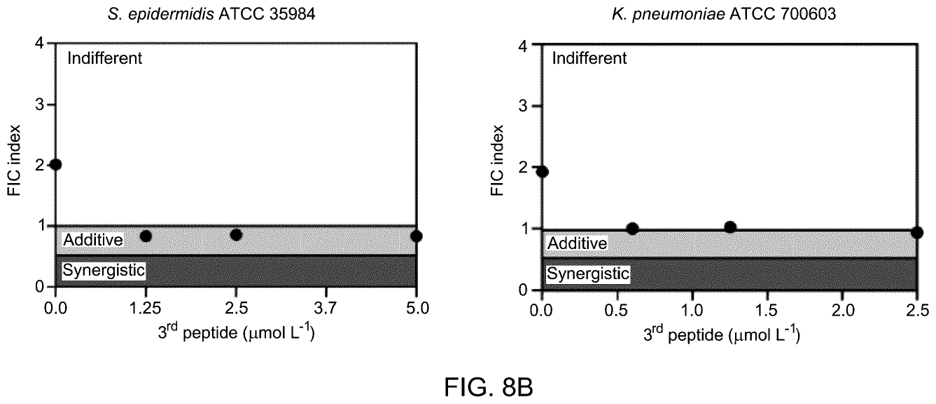

The ability of ApoB encrypted peptides to potentiate each other's activity through synergistic interaction assays was also assessed. Combinations of 2 or 3 peptides were investigated (FIGS. 8A and 8B). Indifferent interactions were obtained for combinations between two peptides (FIG. 7A). Interestingly, combinations among three peptides led to decreased FIC values indicative of additive interactions (FIG. 8B).

Longitudinal killing assays were carried out to evaluate the efficacy over time of the most promising combinations of antimicrobials. A longer exposure to antimicrobials is generally associated with an increased likelihood of selecting for bacterial resistance. 36 Kinetic killing curves were obtained by concomitantly treating bacteria with combinations of ApoB-derived encrypted peptides and colistin or polymyxin B. The speed of killing by antimicrobial peptide (AMPs) is usually attributed to different mechanisms of action. Indeed, most AMPs present more than one mechanism of action, conferring an evolutionary advantage over the single mode of action of standard antibiotics. ^{37,38}The two main reasons affecting killing kinetics are: i) membrane dysfunction following disruption of the phospholipid bilayer; and/or ii) interaction with intracellular targets involved in critical cellular processes, such as RNA and DNA replications, and protein synthesis. ³⁹ AMPs are generally potent and rapid bactericidal agents, causing significant bacterial death within 2 hours. ⁴⁰ Consistent with this, peptides r(P)ApoB_{L}^{Pro} and its shorter derivative r(P)ApoB_{S}^{Pro} took 1 and 3 hours, respectively, to completely kill bacterial cultures (FIG. 1D). Again, significant antimicrobial effects were observed at concentrations much lower than those required when these agents were administered alone (FIG. 1C and FIG. 1D). Moreover, combination therapy killed bacteria much more rapidly (within 10 min) than in monotherapy (60 or 180 min) (FIG. 1D).

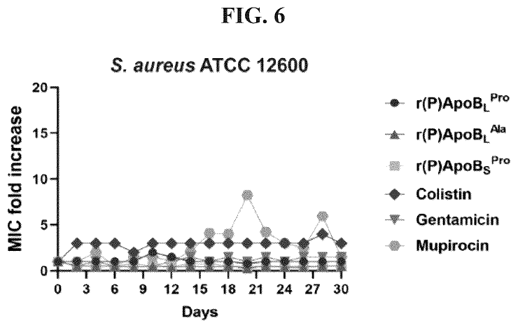

Experiments were performed to assess whether prolonged exposure to ApoB-derived encrypted peptides led to the evolution of resistant phenotypes, since classical AMPs are known to be less likely to trigger bacterial resistance than standard antibiotics. ⁴¹ The Gram-negative bacterial pathogen A. baumannii ATCC 17878, and the Gram-positives S. epidermidis ATCC 35984, and S. aureus ATCC 12600 were longitudinally treated with r(P)ApoB_{L}^{Pro}, r(P)ApoB_{L}^{Ala}, and r(P)ApoB_{S}^{Pro}. Control sample groups were treated with the antibiotics colistin, gentamicin, and mupirocin, selected based on their distinct mechanisms of action, the former destabilizing the bacterial extracellular membrane and the latter acting on intracellular targets. After 30 days of treatment, the MIC values of colistin and mupirocin increased against A. baumannii from 3.12 to 25 μg mL⁻¹ (2.7 to 21.6 μmol L⁻¹) and from 100 to 400 μg mL⁻¹ (200 to 800 μmol L⁻¹), respectively. The MIC value of gentamicin and ApoB-derived encrypted peptides did not change under the experimental conditions tested over the period of the experiment, indicating that A. baumannii did not develop resistance against either gentamicin or the encrypted peptides. Conversely, when S. epidermidis cells were exposed to gentamicin and mupirocin, MIC values increased from 3.12 to 50 μg mL⁻¹ (16.5 to 104.7 μmol L⁻¹) and from 0.0015 to 0.012 μg mL⁻¹ (0.003 to 0.024 μmol L⁻¹). No significant resistance development was observed for S. epidermidis when treated with ApoB-derived encrypted peptides or colistin (FIG. 3C). A. baumannii resistance towards colistin increased by up to 8-fold, whereas S. epidermidis resistance to gentamicin and mupirocin increased by up to 16- and 8-fold, respectively (FIG. 3C). Moreover, when we treated S. aureus ATCC 12600 with each peptide or the selected antibiotics, no variations in the MIC values were observed (FIG. 6) except for mupirocin, whose MIC value increased 8-fold from 0.024 to 0.19 μg mL⁻¹ (0.048 to 0.38 μmol L⁻¹) between days 15 and 21 of treatment (FIG. 6). Importantly, A. baumannii, S. epidermidis, and S. aureus ATCC 12600 did not develop resistance to the encrypted peptides under our experimental conditions (FIG. 3C and FIG. 6), thus underlining their promise as antimicrobials that do not readily select for bacterial resistance. SEM results also revealed that the strains that evolved resistance displayed different morphologies than their antibiotic-susceptible predecessors. For example, both colistin-resistant A. baumannii ATCC 17878 and gentamicin-resistant S. epidermidis ATCC 35984 presented wrinkled borders (FIG. 2E) as opposed to the smooth colonies' characteristic of their morphology at the beginning of the experiment (FIG. 2E).

### Example 2—Anti-biofilm Effects of ApoB-derived Encrypted Peptides

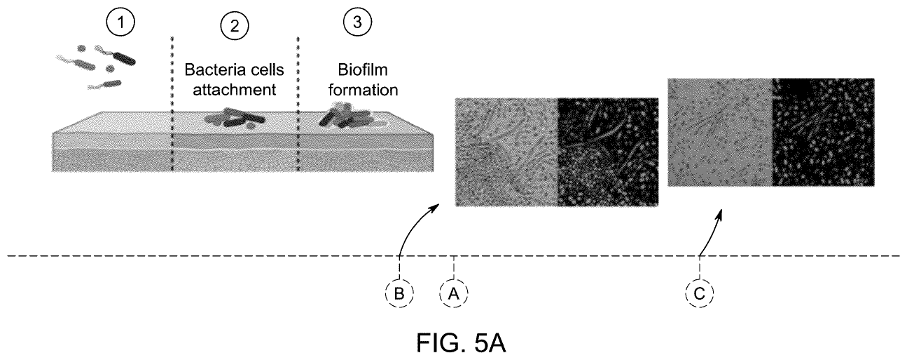

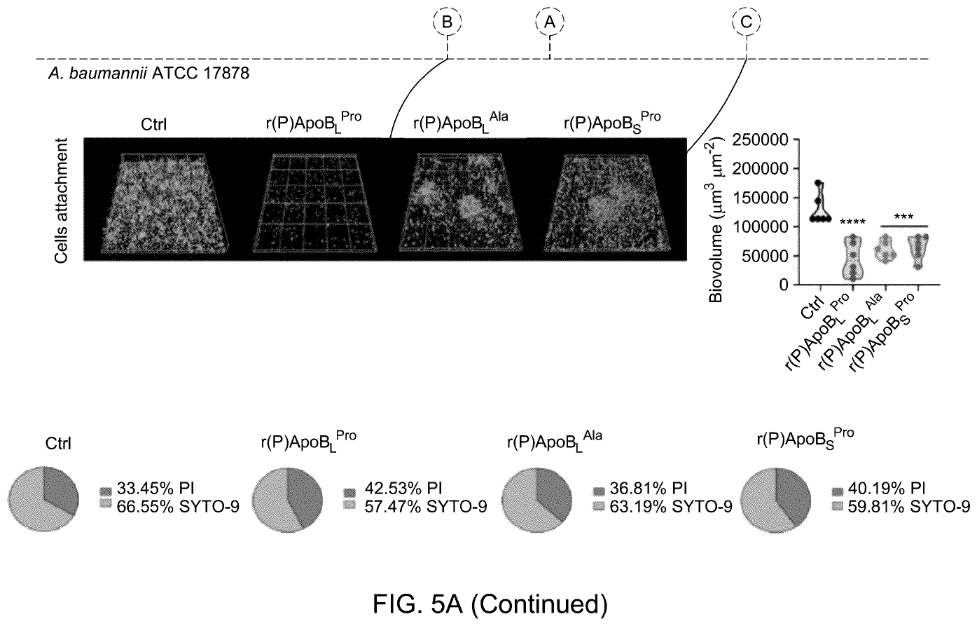

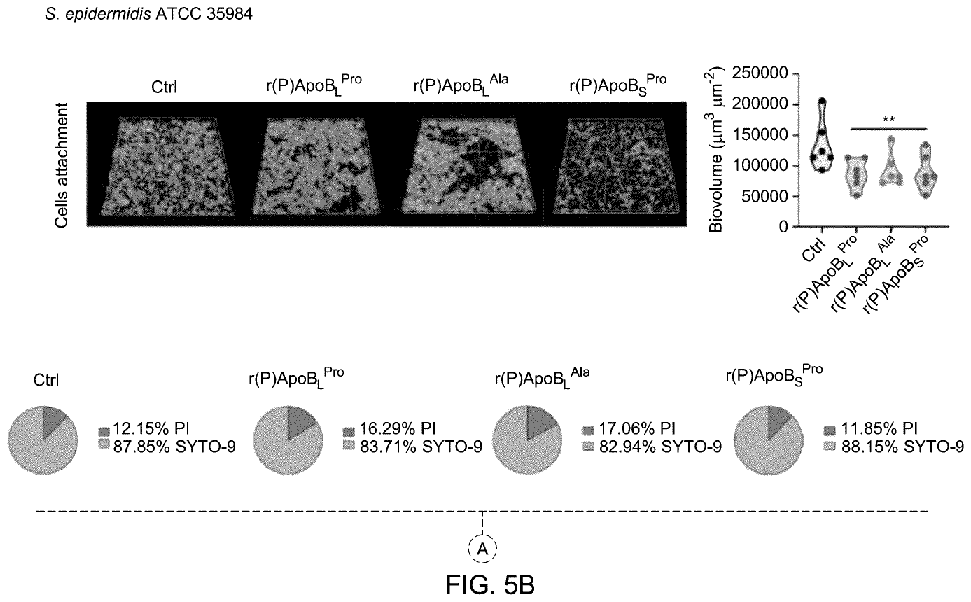

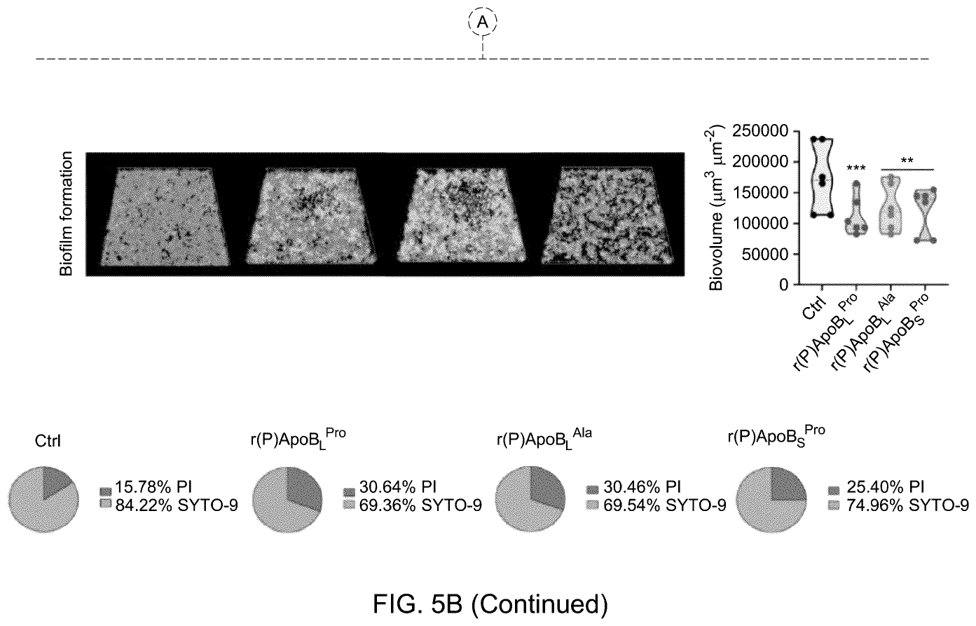

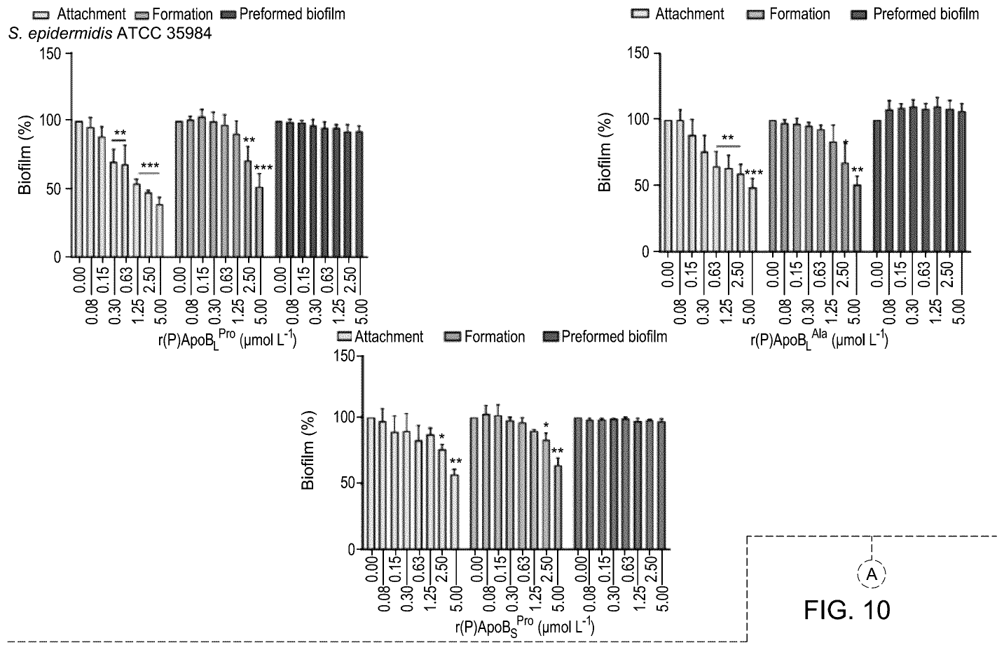

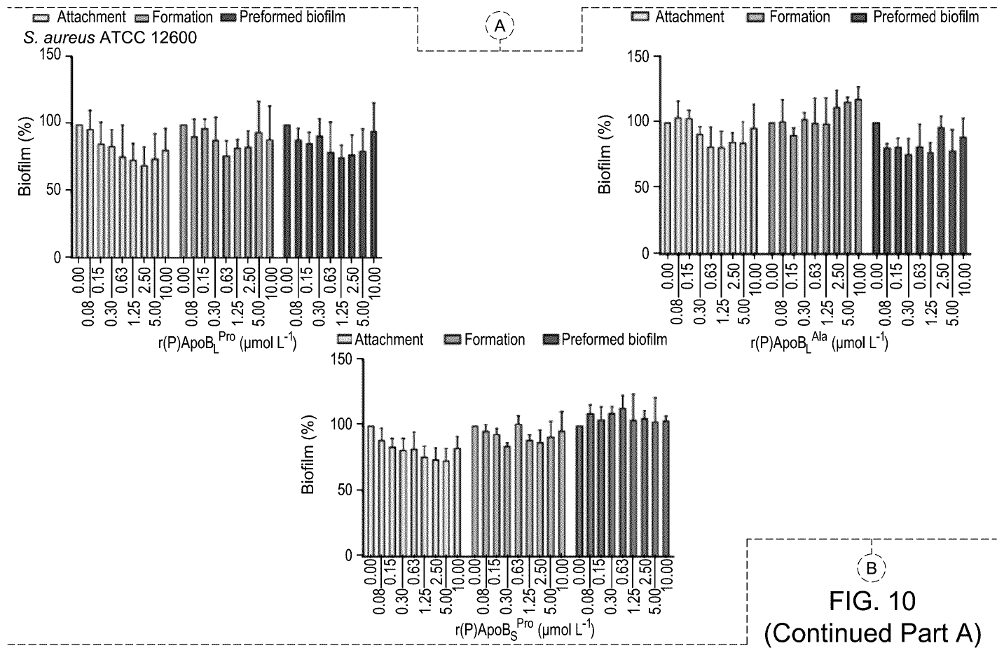

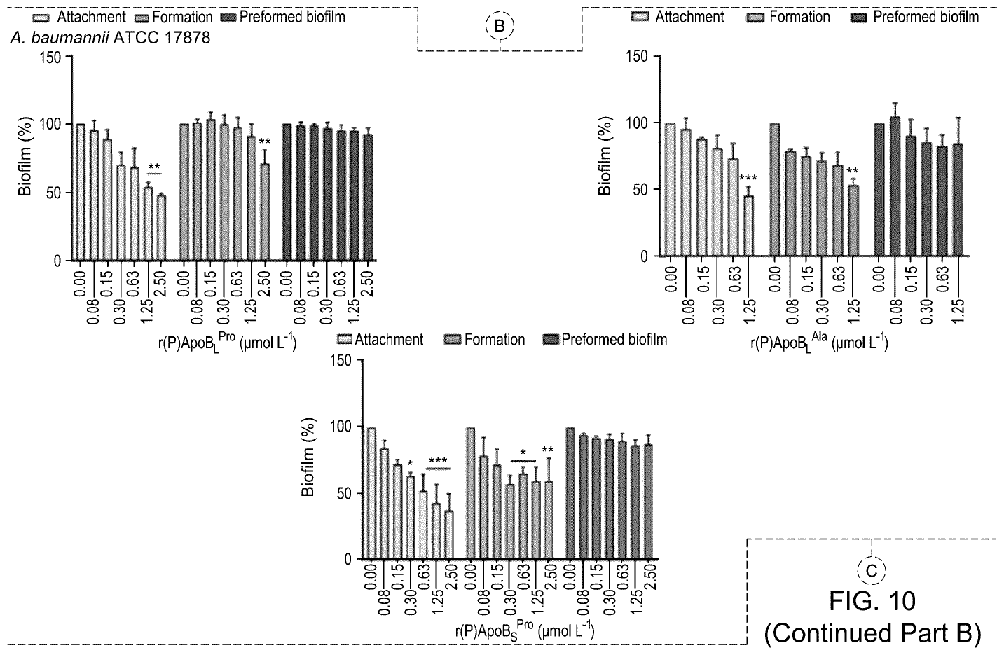

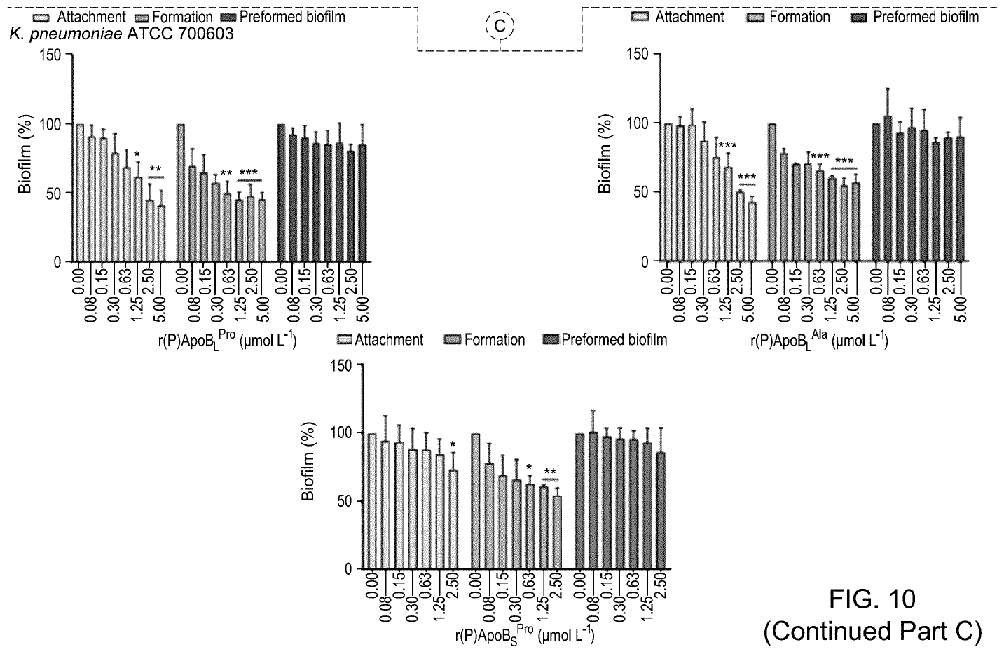

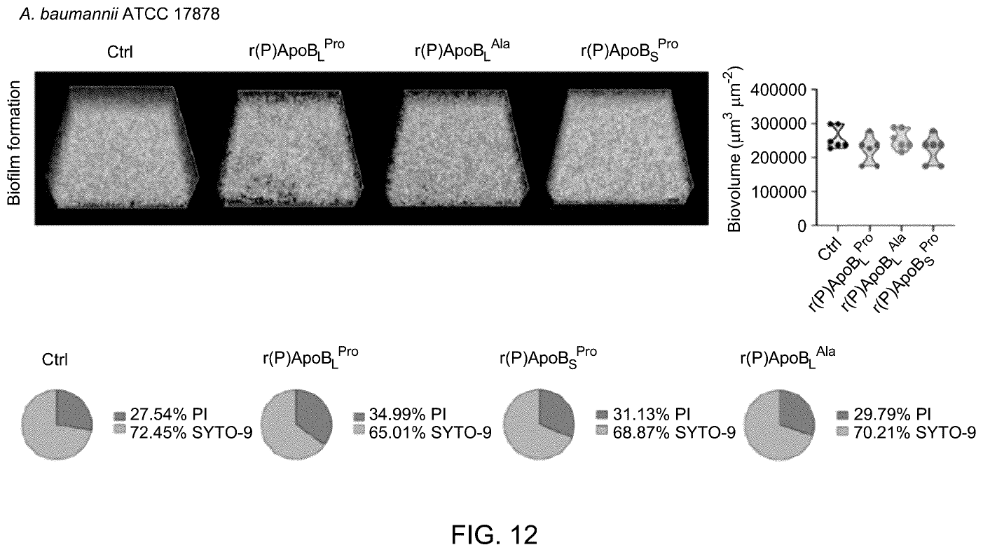

Crystal violet assays were assessed to investigate whether ApoB-derived encrypted peptides prevented biofilm formation of common skin bacterial pathogens, such as S. epidermidis ATCC 35984, S. aureus ATCC 12600, A. baumannii ATCC 17878 and K. pneumoniae ATCC 700603. The effects of the peptides on the three main stages of biofilm development, i.e., adhesion, formation, and preformed biofilm ⁴² were analyzed using the crystal violet assay. ⁴³Sub-MIC peptide concentrations ranging from 1.25 to 5 μmol L⁻¹ led to significant inhibition (30-40%) of biofilm adhesion and formation in the Gram-negative strains A. baumannii ATCC 17878 and K. pneumoniae ATCC 700603, and in the Gram-positive bacterium S. epidermidis ATCC 35984. Conversely, no significant effects were detected on biofilm adhesion and formation of S. aureus ATCC 12600 or preformed biofilms of any of the pathogens tested (FIG. 10). Confocal laser scanning microscopy (CLSM) experiments were also performed to assess the effects of each peptide against A. baumannii ATCC 17878 and S. epidermidis ATCC 35984. The peptides significantly altered biofilm architecture and reduced biofilm biovolume in both strains. Peptide r(P)ApoB_{L}^{Pro} was found to exert the strongest effect (biovolume reduced by 3-fold compared to the untreated control) against A. baumannii ATCC 17878 initial biofilm attachment, whereas r(P)ApoB_{L}^{Ala} and r(P)ApoB_{S}^{Pro} triggered cell filamentation in A. baumannii, suggesting that they may interfere with cell division mechanisms by blocking septation, ¹³consistent with the high percentage of dead cells observed (FIG. 5A). No significant effects for any of the peptides were observed on A. baumannii biofilm formation (FIG. 12). However, the three ApoB-derived peptides affected the biofilm matrix and reduced biofilm biovolume of S. epidermidis ATCC 35984 at sub-MIC concentrations (5 μmol mL⁻¹). Peptides r(P)ApoB_{L}^{Pro} and r(P)ApoB_{L}^{Ala} further affected biofilm formation by inducing cell death (i.e., red cell aggregates in FIG. 5B). Overall, these data suggest that ApoB-derived encrypted peptides exert their anti-biofilm activity against S. epidermidis ATCC 35984 through multiple mechanisms (FIG. 5B). The anti-biofilm activity of these molecules is comparable to that of human hepcidin 20, an AMP secreted by hepatocytes that, at concentrations ranging from 3.15 to 25 μmol L⁻¹, reduces the extracellular matrix mass, alters biofilm architecture, and targets polysaccharide intercellular adhesin in S. epidermidis. ⁴⁴

### Example 3—Biocompatibility and Anti-inflammatory Effects of Encrypted Peptides

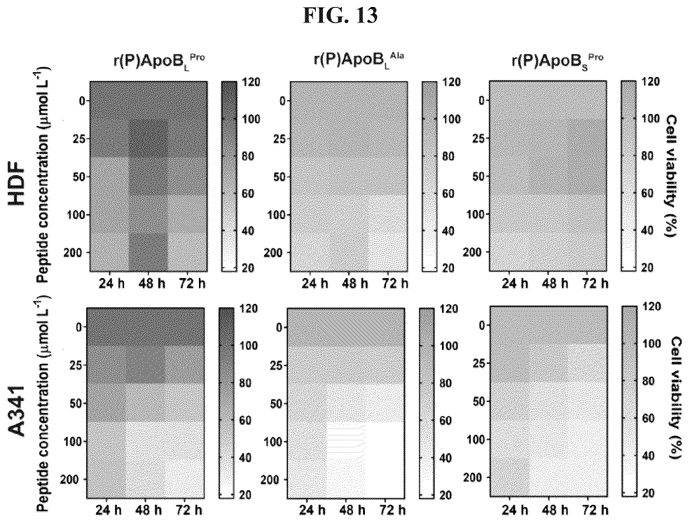

Cytokine-mediated immunogenicity is one potential side effect of peptide therapeutics as it can lead to cytotoxic effects and allergic responses. ^{21,45} To determine whether the peptides exerted any toxic effects against eukaryotic cells and to verify their biocompatibility towards skin cell cultures, dose-response and time-course 3-(4, 5-dimethylthiazolyl-2)-2,5-diphenyltetrazolium bromide (MTT) and lactate dehydrogenase (LDH) cytotoxicity assays were performed against both human dermal fibroblasts (HDF) and human epidermoid carcinoma cell (A431) lines. Due to their cationic nature, most AMPs preferentially interact with negatively charged membranes, such as those of bacteria. The presence of cholesterol and the absence of acidic phospholipids in normal human cell membranes are at the basis of AMPs selective toxicity towards prokaryotic cells. ⁴⁶ Conversely, the net negative charge of tumoral cell membranes, conferred by modifications in phospholipid composition, promotes the anticancer activity of some AMPs. ⁴⁷⁻⁴⁹ When incubated with normal human fibroblasts, ApoB-derived encrypted peptides exhibited mild toxicity (up to 20-30% decrease in cell viability) after 72 h of peptide treatment at 20 μmol L⁻¹. All peptides at 10-20 μmol L⁻¹ decreased carcinoma cell viability by 30-40% after 48-72 h (FIG. 7B). r(P)ApoB_{S}^{Pro} reduced tumoral cell viability by 30% after 24 h (FIG. 7B). To further analyze peptide biosafety towards normal and tumoral skin cells, the peptides were also tested at higher concentrations (25; 50; 100; 200 μmol L⁻¹) (FIGS. 13). At 100 and 200 μmol L⁻¹, ApoB encrypted peptides decreased cell viability up to 30-40% of normal human fibroblasts and over 70% of human epidermoid carcinoma cells (FIG. 13). These data confirm that the peptides are safe against normal cells at the doses required to exert antimicrobial properties. To evaluate whether peptides exert cytostatic or cytotoxic effects through membrane damage, LDH release in culture medium was detected as a biomarker of membrane damage. ⁵⁰ Thus, human dermal fibroblasts (HDF) and human epidermoid carcinoma cells (A431) were treated with each peptide at a concentration of 10 and 20 μmol L⁻¹ for 72 h. No significant LDH release was detected, thus indicating no damage to eukaryotic cell membranes caused by exposure to the peptides (FIG. 7C).

Enzyme-linked immunosorbent (ELISA) assays were also performed to exclude any potential undesired inflammatory response triggered by treating human differentiated monocytes (THP-1) with the ApoB-derived encrypted peptides. The levels of monocyte chemoattractant protein-1 (MCP-1), interleukin 8 (IL-8), and tumor necrosis factor (TNFα) were assessed in differentiated THP-1 cells upon treatment with each peptide at 5 or 20 μmol L⁻¹ for 24 h. No significant release of the pro-inflammatory cytokines IL-8 and TNFα, or the chemokine MCP-1, which is involved in leukocyte activation and migration, was detected in peptide treated groups compared to positive control samples incubated with 10 ng mL⁻¹ of lipopolysaccharide (LPS from P. aeruginosa PAO1) (FIG. 17). ¹² Clinical studies have revealed that several skin diseases are associated with an increased inflammatory response induced by endogenous AMPs such as defensins and the cathelicidin LL-37, both of which trigger secretion of several cytokines at the injury site. ⁵¹ ApoB-derived encrypted peptides operated differently, as they did not increase cytokine release.

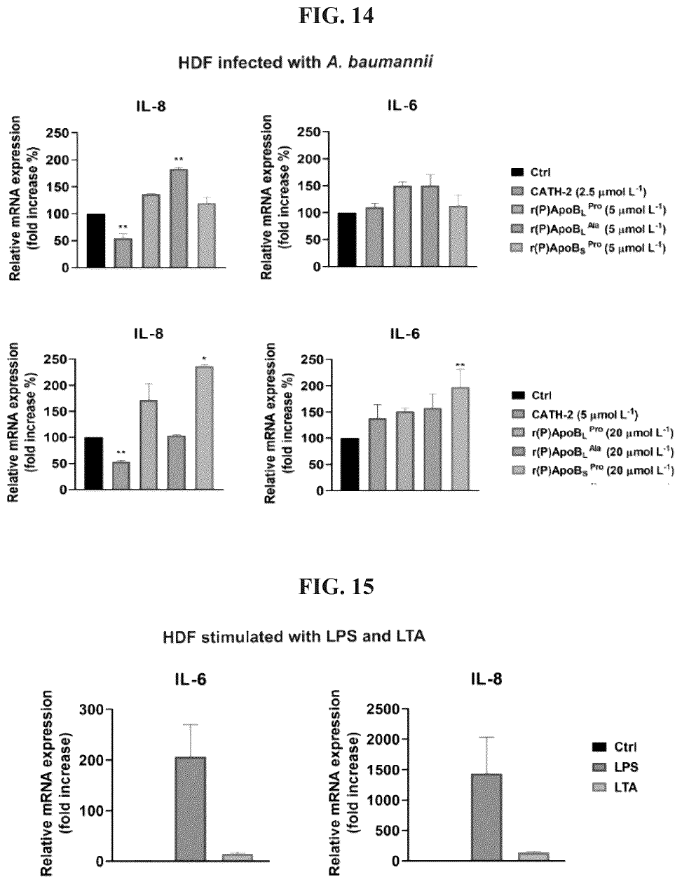

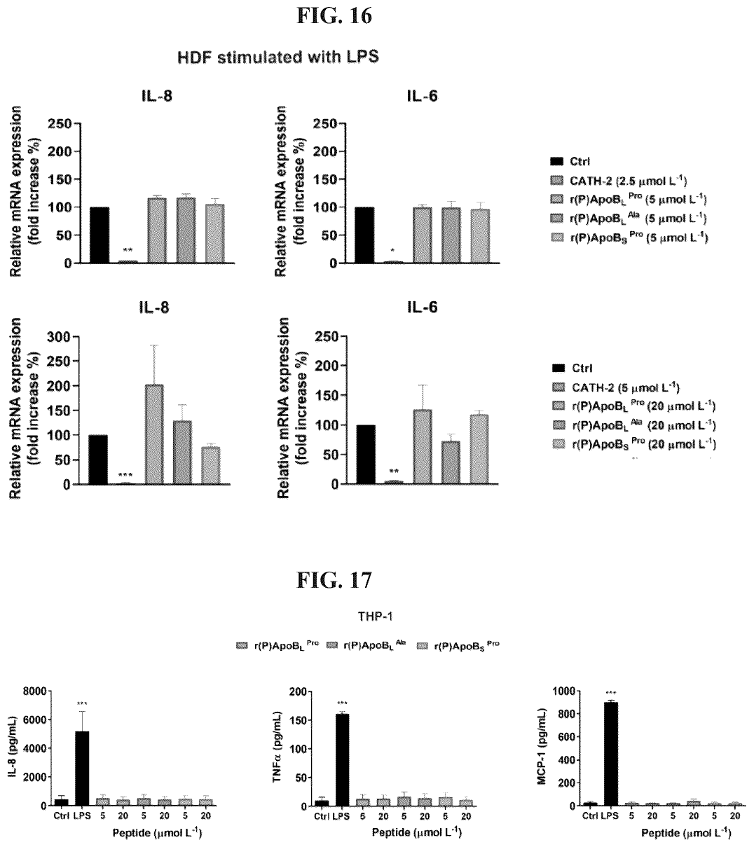

Skin infections caused by opportunistic bacterial pathogens, such as Staphylococci, Acinetobacter, and Pseudomonas strains typically trigger the activation of a significant immune response in the underlying skin cells. ⁵² Recent studies demonstrated that S. aureus contributes to long-lasting cutaneous inflammation and local immunosuppression. ⁵³ For this reason, an evaluation was also performed on the effects of ApoB-derived peptides on HDFs infected with S. aureus ATCC 12600, the primary pathogen infecting skin and soft tissues. ⁵⁴ Using RT-qPCR, the effects of ApoB-derived encrypted peptides (10 μmol L⁻¹) and the positive control peptide CATH-2 (2.5 μmol L⁻¹) were assessed on the expression of pro-inflammatory cytokines IL-8 and IL-6 (FIG. 7D). Peptide r(P)ApoB_{S}^{Pro} reduced the inflammatory response triggered by HDFs infection with S. aureus ATCC 12600 (FIG. 7D). It is worth noting that patients with psoriasis, a skin disorder associated with infections caused by streptococci and Staphylococcus species, tend to have reduced ApoB plasma levels, thus indicating an interesting and complex role of this lipoprotein in host defense. ⁵⁵ The results (FIG. 7D) were in line with prior studies describing the role of ApoB in controlling S. aureus virulence. ⁵⁶ Conversely, no anti-inflammatory effects were observed (FIG. 14) when ApoB encrypted peptides (at 5 and 20 μmol L⁻¹) were tested in HDF cells infected with A. baumannii ATCC 17878. However, in the same conditions, r(P)ApoB_{L}^{Ala} and r(P)ApoB_{S}^{Pro} peptides (at 5 and 20 μmol L⁻¹, respectively) were found to increase the expression levels of IL-8 and IL-6 (FIG. 14). Stimulation of HDF with LPS from E. coli increased the expression levels of IL-8 and IL-6 compared to the same cells stimulated with LTA from S. aureus (FIG. 15). When ApoB encrypted peptides [r(P)ApoB_{L}^{Pro}, r(P)ApoB_{L}^{Ala} and r(P)ApoB_{S}^{Pro}] were tested on HDF stimulated with LPS, they did not display any anti-inflammatory or pro-inflammatory effects (FIG. 16).

Histone deacetylase (HDAC) enzymes, encoded by the HDAC genes, play a key role in integrating commensal bacteria-derived signals to calibrate epithelial cell responses. ⁵⁷ Consequently, a decrease in HDAC activity in skin keratinocytes correlates with imbalances in physiological host-commensal interactions. Thus, to rule out any potential side effect of the peptides against skin cells, it was assessed whether r(P)ApoB_{L}^{Pro}, r(P)ApoB_{L}^{Ala}, and r(P)ApoB_{S}^{Pro} affected the activity of HDAC enzymes. Briefly, HaCaT human keratinocytes were treated for 30 min with 20 μmol L⁻¹ of each peptide and 50 nmol L⁻¹ of the selective HDAC inhibitor trichostatin A, which was used as a positive control. ApoB-derived encrypted peptides did not affect HDAC activity, indicating that they may not negatively influence the balance between skin microbiota and epithelial cells (FIG. 7E).

### Example 4—In Vitro and In Vivo Antimicrobial Activity of Synthetic Retro-inverso Peptide r(P)ApoB_{S}^{Pro}

One of the main limitations hindering the development of peptide therapeutics is their low stability in complex biological environments that contain proteolytic enzymes. ⁵⁸ Linear peptides are sensitive to proteolysis, drastically reducing their biological activity and their application as antimicrobial agents. ⁵⁹

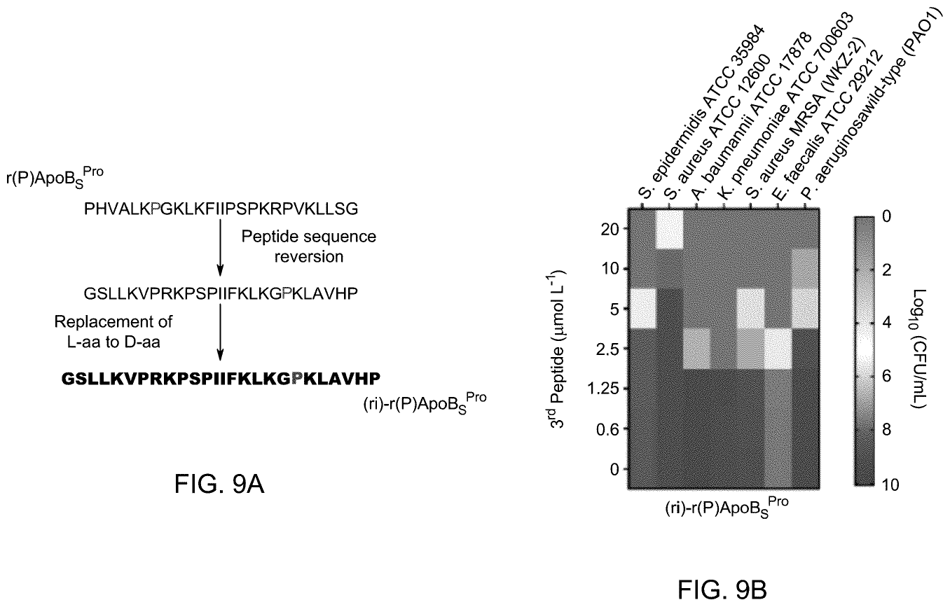

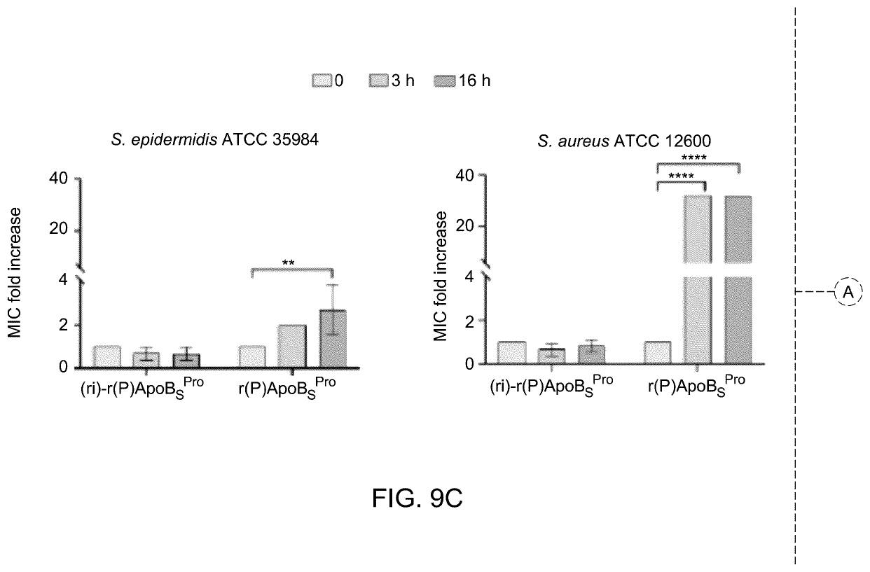

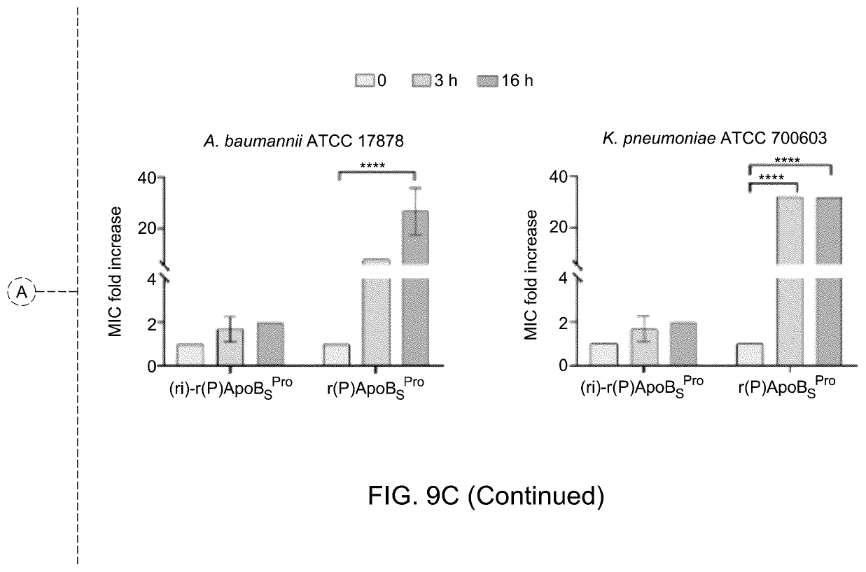

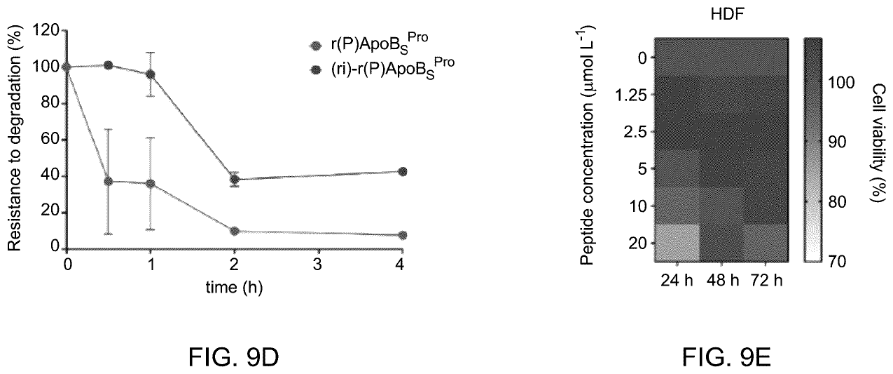

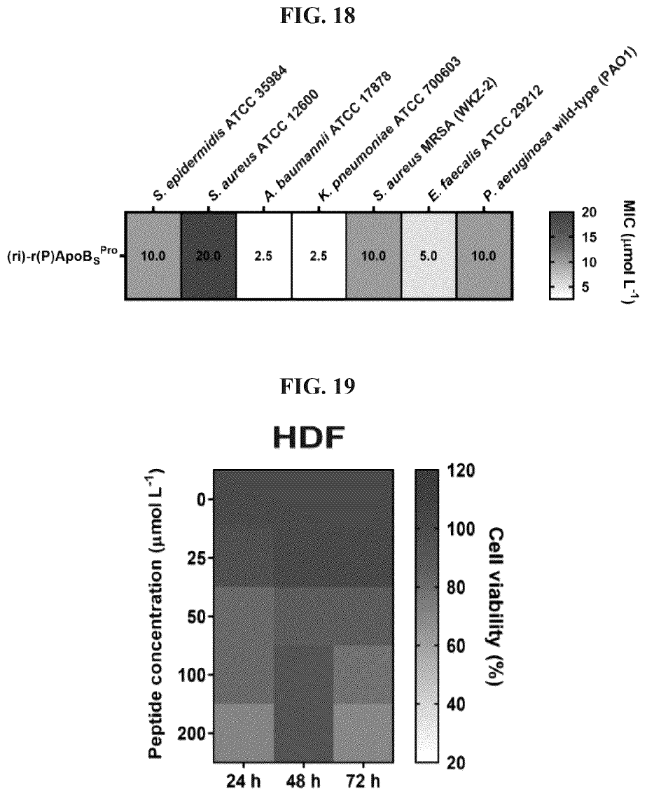

Peptide r(P)ApoB_{S}^{Pro} was selected as a lead compound derived from ApoB-100 because of its shorter length and excellent anti-infective and cytotoxic profiles (FIGS. 1B and 1C; Table S1; FIG. 7B). The retro-inverso of r(P)ApoB_{S}^{Pro} [.e., (ri)-r(P)ApoB_{S}^{Pro}] was prepared by reversing its peptide sequence and replacing all (L) amino acids for their (D) counterparts (FIG. 9A). First, the antimicrobial activity of (ri)-r(P)ApoB_{S}^{Pro} was evaluated against seven pathogens. Peptide (ri)-r(P)ApoB_{S}^{Pro} displayed increased activity against Gram-negative rather than Gram-positive bacterial strains, as previously reported for its parent peptide r(P)ApoB_{S}^{Pro} (FIG. 9B, FIG. 18, and Table S3).

TABLE S3
MIC values (μmol L⁻¹) of (ri)-r(P)ApoB_{S}^{Pro}. Antimicrobial
activity of (ri)-r(P)ApoB_{S}^{Pro} against S. epidermidis ATCC 35984,
S. aureus ATCC 12600, A. baumannii Pro ATCC 17878, K. pneumoniae
ATCC 700603, S. aureus MRSA (WKZ-2), E. faecalis ATCC 29212
and P. aeruginosa wild-type PAO1 strains.

| | MIC (μmol L⁻¹) |
|---|---|
| Bacterial Strains | (ri)-r(P)ApoB_{S}^{Pro} |
| S. epidermidis ATCC 35984 | 10 |
| S. aureus ATCC 12600 | 20 |
| A. baumannii ATCC 17878 | 2.5 |
| K. pneumoniae ATCC 700603 | 2.5 |
| S. aureus MRSA (WKZ-2) | 10 |
| E. faecalis ATCC 29212 | 5 |
| P. aeruginosa wild-type (PAO1) | 10 |

The MIC values of (ri)-r(P)ApoB_{S}^{Pro} ranged from 2.5 to 5 μmol L⁻¹ against Acinetobacter baumannii ATCC 17878, Klebsiella pneumoniae ATCC 700603 and Enterococcus faecalis ATCC 29212, whereas it was active at 10 μmol L⁻and 20 μmol L⁻¹ against Staphylococcus epidermidis ATCC 35984, Staphylococcus aureus ATCC 12600, Staphylococcus aureus MRSA (WK7-2) and Pseudomonas aeruginosa wild-type (PAO1), respectively, thus demonstrating that the synthetic modifications did not alter the antimicrobial profile of the original peptide.

To determine its proteolytic stability, it was assessed whether (ri)-r(P)ApoB_{S}^{Pro} was less susceptible to serum proteases compared to its parent peptide r(P)ApoB_{S}^{Pro}. The MIC values of the peptide were measured upon incubation in 10% fetal bovine serum, which contains human endo- and exoproteases, for 1 and 16 hours. ⁶² Whereas natural r(P)ApoB_{S}^{Pro} peptide completely lost its activity upon incubation in serum showing MIC values against Staphylococcus aureus ATCC 12600, Acinetobacter baumannii ATCC 17878 and Klebsiella pneumoniae ATCC 700603 of >80 μmol L⁻¹, as opposed to 5-20 μmol L⁻¹ in regular medium (FIG. 9C), the antimicrobial activity of (ri)-r(P)ApoB_{S}^{Pro} retro-inverso peptide remained constant even after 16 hours of pre-incubation in serum (FIG. 9C). These antimicrobial activity results corroborate the degradation profile data obtained from analyzing aliquots of the peptide solution exposed to serum by using mass spectrometry coupled to liquid chromatography (FIG. 9D). Indeed, the natural parent peptide degraded after 30 minutes of exposure to serum proteases whereas (ri)-r(P)ApoB_{S}^{Pro} demonstrated increased resistance by persisting (˜50% of initial concentration added) 4 h post-exposure to proteases in serum (FIG. 9D). These results demonstrate the high stability of the inventive engineered peptides when compared with previously described peptides in the literature. ⁶³⁻⁶⁶ On the contrary, the other peptides composed of L-amino acids [i.e., r(P)ApoB_{L}^{Pro}, r(P)ApoB_{L}^{Ala} and r(P)ApoB_{S}^{Pro}] were not stable in the presence of serum and degraded within minutes (FIG. 9D), thus further underscoring the increased stability of our retro-inverso variant.

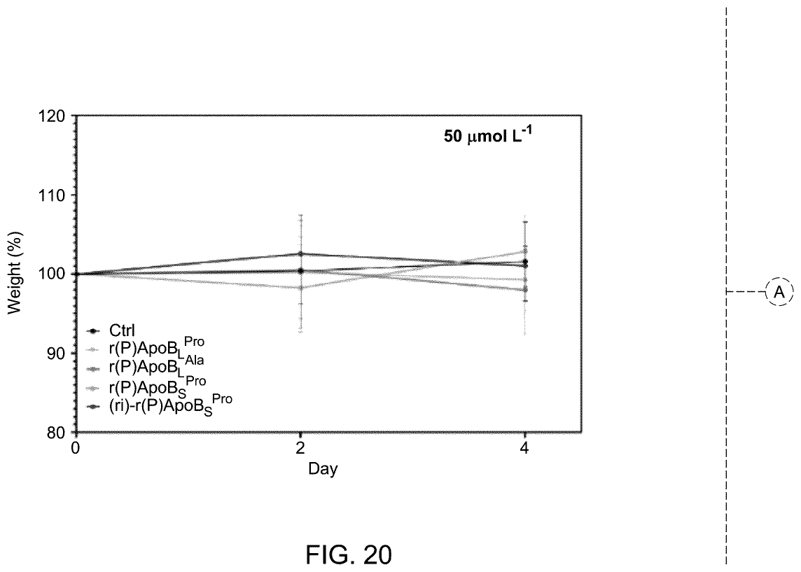

Additional experiments were performed to verify the biocompatibility of (ri)-r(P)ApoB_{S}^{Pro} towards skin cell cultures, since incorporating D-amino acids into peptide sequences may lead to toxic effects. ⁶⁷ In dose-response and time-course MTT cytotoxicity assays, the peptide (ri)-r(P)ApoB_{S}^{Pro} decreased human fibroblasts (HDFs) viability by 20-30% at the highest concentrations tested (100 and 200 μmol L⁻¹) (FIG. 9E and FIG. 19). Similar to r(P)ApoB_{S}^{Pro}, the retro-inverso peptide displayed lower toxic effects toward HDFs than r(P)ApoB_{L}^{Pro} and r(P)ApoB_{L}^{Ala} (FIG. 9E and FIG. 13). These data suggest that the shorter peptides [r(P)ApoB_{S}^{Pro} and (ri)-r(P)ApoB_{S}^{Pro}] present decreased toxicity compared to the longer peptide versions (FIG. 7B and FIG. 9E; FIG. 13 and FIG. 19). Altogether, these results demonstrate that the substitution of L-amino acids by D-amino acids significantly increased peptide stability without altering its antimicrobial and cytotoxic activities, thus increasing the translational potential of this agent.

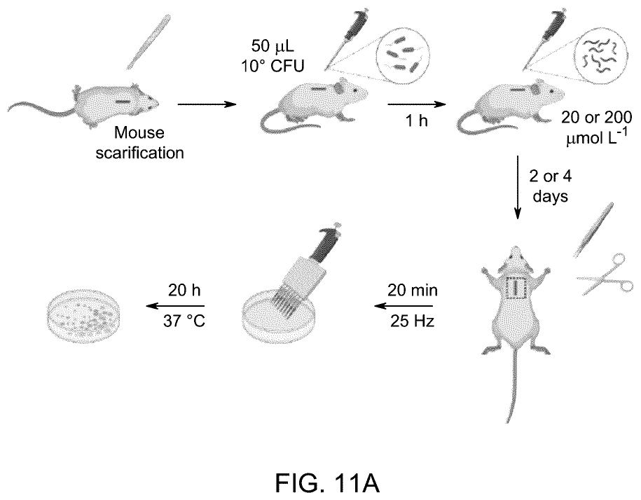

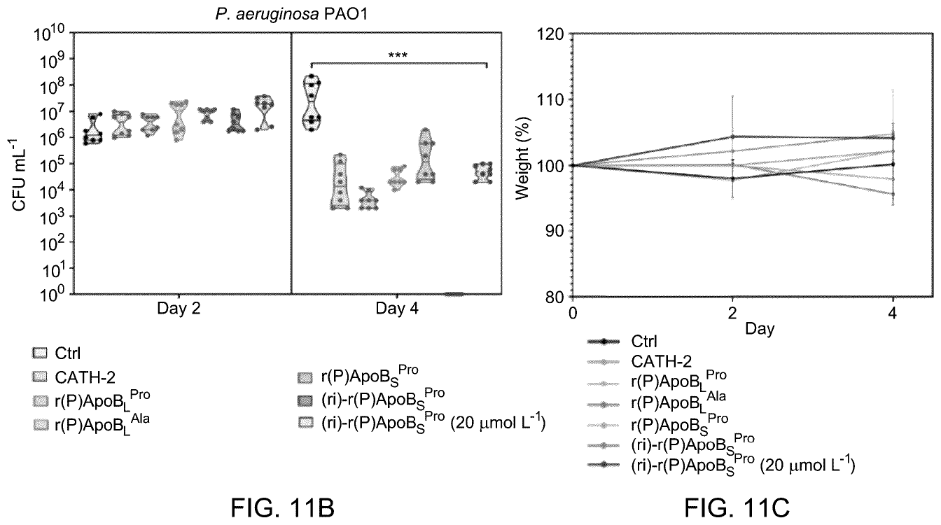

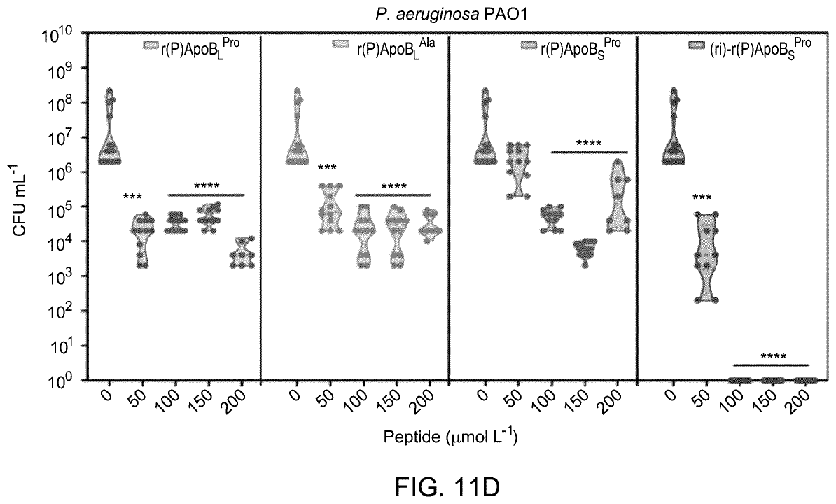

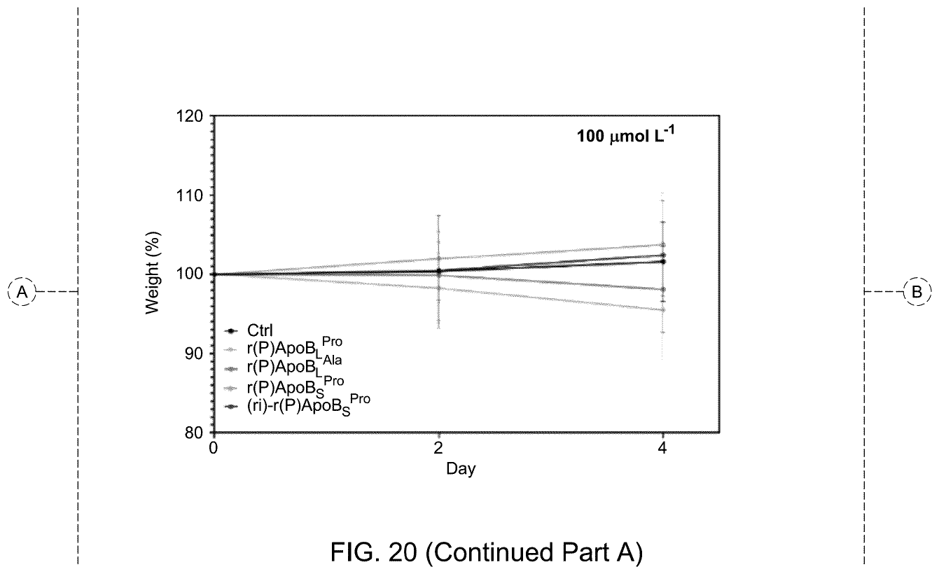

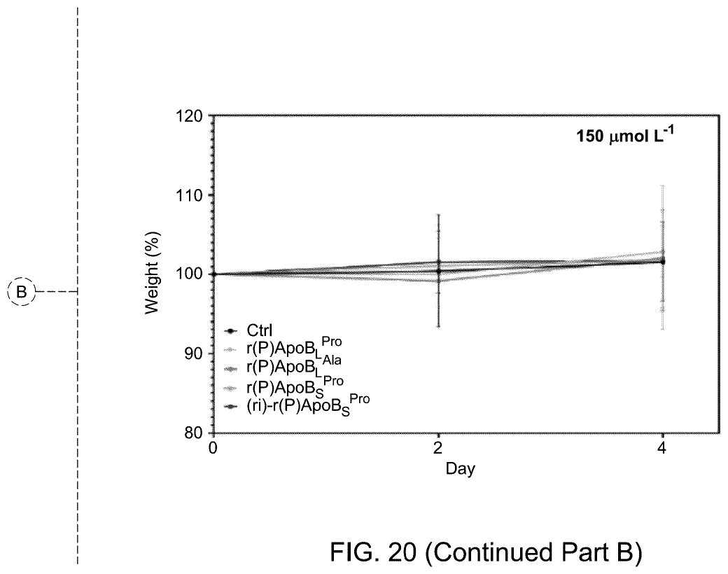

To assess the anti-infective potential of the peptides in vivo, a murine abscess infection model was used (FIG. 11). The Gram-negative bacterium Pseudomonas aeruginosa PAO1, which is responsible for dangerous skin infections, ⁶⁸ was susceptible to all ApoB encrypted peptides (FIG. 1B and FIG. 9B) and thus utilized in the infection model. Upon induction of skin infection in mice, a single dose of either (ri)-r(P)ApoB_{S}^{Pro} (at 20 or 200 μmol L⁻¹) or each of the three natural ApoB-derived encrypted peptides (200 μmol L⁻¹) was administered. Treatment with r(P)ApoB_{L}^{Pro}, r(P)ApoB_{L}^{Ala}, and r(P)ApoB_{S}^{Pro} (all at 200 μmol L⁻¹) significantly reduced P. aeruginosa colony-forming unit (CFU) counts by ˜3 to 4 orders of magnitude after four days of treatment. The tissue was homogenized, and the bacterial load was quantified through CFU count assays as this quantitative method accurately reflects the number of the bacteria present in a given infected area. ⁶⁹ Similar results were obtained with our control peptide CATH-2. Importantly, treatment with (ri)-r(P)ApoB_{S}^{Pro} at a significantly lower dose (20 μmol L⁻¹) reduced bacterial loads by ˜3 orders of magnitude, and treatment with a higher peptide concentration (200 μmol L⁻¹) completely sterilized the infection four days post-treatment (FIG. 11B). Peptide (ri)-r(P)ApoB_{S}^{Pro} exerted its anti-infective activity in a dose-dependent manner (FIG. 11D). Four-days post-treatment, the retro-inverso peptide reduced bacterial counts by 3 orders of magnitude at 20 and 50 μmol L⁻¹ (FIG. 11D) and completely eradicated P. aeruginosa infections at concentrations higher than 100 μmol L⁻¹ (FIG. 11D). No significant weight changes or any obvious inflammation events were detected during the treatment period, thus confirming the lack of toxicity of these peptides in our murine model (FIG. 11C and FIG. 20). Altogether, the data demonstrate the anti-infective activity of (ri)-r(P)ApoB_{S}^{Pro} in a pre-clinical mouse model.

### Methods

Materials. Unless specified otherwise, all reagents used in the present study were purchased from Sigma-Merck (Milan, Italy).

Bacterial strains and growth conditions. All bacterial strains used in the analyses, i.e., S. epidermidis ATCC 35984, S. aureus ATCC 12600, A. baumannii ATCC 17878, K. pneumoniae ATCC 700603, S. aureus MRSA (WK7-2), E. faecalis ATCC 29212 and Pseudomonas aeruginosa PAO1, were grown in the same media and experimental conditions as previously reported. ^{5,6,71}

Peptides. Expression and isolation of recombinant peptides were carried out as previously described. ¹⁹ CATH-2 and (ri)-r(P)ApoB_{S}^{Pro} peptides were obtained from CPC Scientific Inc. (Sunnyvale, USA) and CASLO ApS (Kongens Lyngby, Denmark), respectively.

Antimicrobial activity. The antimicrobial activity of ApoB-derived encrypted peptides was assessed against a panel of skin pathogens, such as S. epidermidis ATCC 35984, S. aureus ATCC 12600, A. baumannii ATCC 17878, K. pneumoniae ATCC 700603, S. aureus MRSA (WK7-2), E. faecalis ATCC 29212 and P. aeruginosa PAO1 by using the broth microdilution method. ²⁰ Bacteria were grown to mid-logarithmic phase in MHB at 37° C. Then, cells have been diluted to 4×10⁶ CFU/mL in Difco 0.5× Nutrient Broth (Becton-Dickenson, Franklin Lakes, N.J.) and mixed 1:1 v/v with two-fold serial dilutions peptides (0-40 μmol L⁻¹). Following incubation over-night, each sample was diluted, plated on TSA, and incubated at 37° C. for 24 h to count the number of colonies. All experiments were performed in three independent replicates.

Antimicrobial activity of peptides upon pre-incubation in 10% serum. The antimicrobial activity of r(P)ApoB_{S}^{Pro} and (ri)-r(P)ApoB_{S}^{Pro} was evaluated against four bacterial strains upon pre-incubation in 10% FBS (fetal bovine serum, Microgem Lab, Cat. 51860, Italy). All the peptides were incubated for 1 h or 16 h in serum at 37° C. (water bath) prior to MIC value determination by standard protocols. ²⁰ Experiments were carried out in triplicate for each peptide.

Stability assay. The resistance to enzymatic degradation assay was performed according to the method described by Powell et al. ⁷² Briefly, peptides at 2 mg mL⁻¹ were exposed to a solution of 25% fetal bovine serum in water. Aliquots were collected after 0.5, 1, 2, and 4 h and 10 μL of trifluoroacetic acid was added to them while the samples were on ice for 10 min. The enzymatic degradation of peptides was followed by reverse-phase high performance liquid chromatography coupled to mass spectrometry (RP-HPLC/ESI-MS). The percentage of remaining peptide was calculated by integrating the area under the curve related to the peptide at time point zero.

$${[ Peptide ]}_{remaining} = 100 \times \frac{{AUC}_{t}}{{AUC}_{0}} ,$$

where AUC_{X} is the area under the curve of the peak related to the peptide after t h (t=0.5, 1, 2, and 4 h), and AUC₀ is the area under the curve of the peak related to the peptide at the beginning of the experiment. Three independent experimental replicates were performed, as previously described. ⁷³

DiSC₃(5) assay. Three independent cytoplasmic membrane depolarization assays were carried out on S. epidermidis ATCC 35984, S. aureus ATCC 12600, A. baumannii ATCC 17878 and K. pneumoniae ATCC 700603 using the 3,3′-dipropylthiadicarbocyanine iodide (diSC₃-5-TCI America), which is a membrane potential-sensitive dye. ²³ Bacterial cells were grown to mid-logarithmic phase and then washed and resuspended in 5 mmol L⁻¹ HEPES buffer (pH 7.2) containing 0.1 mol L⁻¹ KCl and 20 mmol L⁻¹ glucose at a density corresponding to an optical value at 600 nm of 0.06-0.03. The cell suspension was then incubated with 1 μmol L⁻¹ DiSC₃ (5) for 45 min to stabilize the fluorescence, and then the peptides were added to bacterial suspensions at concentrations corresponding to their MIC. Changes in fluorescence intensity were continuously recorded by using GloMax® Discover System (Promega, Madison, Wis., USA), with excitation and emission wavelengths of 620 nm and 670 nm, respectively.

NPN assay. The outer-membrane permeability 1-N-phenylnapthylamine (NPN) uptake assay was performed using the Gram-negative pathogens A. baumannii ATCC 17878 and K. pneumoniae ATCC 700603. Bacterial cells were grown to mid-logarithmic phase and then washed and resuspended in 5 mmol L⁻¹ HEPES buffer (pH 7.2) at a density corresponding to an optical value at 600 nm of 0.4. Cell suspensions were incubated with each peptide at a concentration corresponding to its MIC value after which 4 μL of NPN solution (0.5 mmol L⁻¹, working concentration of 10 μmol L⁻¹ after dilutions) was added. Changes in fluorescence intensity were continuously recorded by using a GloMax® Discover System (Promega, Madison, Wis., USA), with excitation and emission wavelengths of 350 nm and 420 nm, respectively. Membrane permeability assays were independently carried out three times.

Checkerboard assay and definition of fractional inhibitory concentration (FIC) index. Combinations of ApoB-derived encrypted peptides and antimicrobial agents were tested on S. epidermidis ATCC 35984, S. aureus ATCC 12600, A. baumannii ATCC 17878, K. pneumoniae ATCC 700603 by the so-called “checkboard” assay to determine Fractional Inhibitory Concentration (FIC) indexes. To this purpose, two-fold serial dilutions of each peptide were tested in combination with two-fold serial dilutions of peptide, EDTA or antibiotics widely used in topical formulations, i.e vancomycin, erythromycin, colistin, polymyxin B, fusidic acid, clindamycin, gentamicin, and benzoyl peroxide. The FIC indexes of two-drug combinations were calculated as follows: FIC_{A}+FIC_{B}, where

$${FIC}_{A} = \frac{MIC of drug A in combination}{MIC of drug A alone} ,$$

$$FI {C}_{B} = \frac{MIC of drug B in combination}{MIC of drug B alone} .$$

In the case of three-drug combinations, we measured the effects of the third drug

$${FIC}_{C} = \frac{MIC of drug C in combination}{MIC of drug C alone}$$

added to the previous two drugs combined (A+B). The FIC indexes of three-drug interactions were calculated as follows:

$$\frac{( F I {C}_{A} + F I {C}_{C} ) + ( F I {C}_{B} + F I {C}_{C} )}{2} .$$

FIC indexes≤0.5 were classified as synergism, FIC indexes between 0.5 and 1 or 1 and 4 were associated with additive and indifferent effects, respectively. ⁵

Scanning electron microscopy analyses. To perform scanning electron microscopy (SEM) analyses, K. pneumoniae ATCC 700603 (2×10⁸ CFU/mL) was incubated with 0.58 μmol L⁻¹ r(P)ApoB_{S}^{Pro} in combination with 0.36 μmol mL⁻¹ colistin for 3 h at 37° C. Following incubation, the samples were processed and characterized as previously reported. ⁷⁴

Killing kinetic studies. To kinetically analyze the anti-bacterial effects of ApoB-derived encrypted peptides co-administrated with conventional antibiotics (e.g., colistin and polymyxin B), experiments were performed using K. pneumoniae ATCC 700603 treated with a combination of both antimicrobials or with the single agents at concentrations corresponding to their MIC. Bacterial cells were diluted to 4×10⁶ CFU/mL in Difco 0.5× Nutrient Broth and mixed 1:1 v/v with the peptide, the antibiotic or both. At defined time points, samples were serially diluted, and each dilution was plated on tryptic soy agar. Following an incubation of 20 h at 37° C., colonies were counted.

Bacterial resistance development assay. S. epidermidis ATCC 35984, S. aureus ATCC 12600 and A. baumannii ATCC 17878 bacterial strains were exposed to colistin, gentamicin, mupirocin, r(P)ApoB_{L}^{Pro}, r(P)ApoB_{L}^{Ala} or r(P)ApoB_{S}^{Pro}. Once, we detected the MIC values for each peptide or antibiotic against the bacterial strains tested, we transferred bacterial cells that survived the exposure at a sub-inhibitory (MIC/2) concentration, and they were re-grown and re-exposed to the respective peptide or antibiotic. ²⁰ The treatment was repeated for 30 days. Strains that developed resistance to antibiotics presented higher MICs at subsequent passages and the cells from the last passage were isolated and stored for scanning electron microscopy analyses.

Anti-biofilm activity assays. Anti-biofilm activity assays were performed on S. epidermidis ATCC 35984, S. aureus ATCC 12600, A. baumannii ATCC 17878, and K. pneumoniae ATCC 700603. Bacteria were grown over-night at 37° C. and then diluted to 4×10⁸ CFU/mL in 0.5×MHB medium. Incubations with increasing concentrations of each peptide (0-40 μmol L⁻¹) were carried out, as previously described, ^{5,75} either for 4 h or 24 h, to test peptide effects on cell attachment or on biofilm formation, respectively. Instead, to evaluate the effect of peptides on preformed biofilm, bacterial biofilm was formed for 24 h at 37° C., and subsequently treated with the peptides. In the case of crystal violet assays, bacterial biofilm was washed with phosphate buffer (PBS 1×) and then incubated with the dye (0.04%) for 20 min at room temperature. At the end of the incubation period, samples were washed with PBS and then the dye bound to cells was dissolved in acetic acid 33%. Spectrophotometric analyses were then carried out at a wavelength of 600 nm. Confocal laser scanning microscopy analyses in static conditions were performed by using the Thermo Scientific™ Nunc™ Lab-Tek™ Chambered Coverglass systems (Thermo Fisher Scientific, Waltham, Mass., USA). The viability of cells embedded within the biofilm structure was assessed by staining the samples using the LIVE/DEAD® Bacterial Viability kit (Molecular Probes Thermo Fisher Scientific, Waltham, Mass., USA) according to manufacturer instructions. A confocal laser scanning microscope (Zeiss LSM 710, Zeiss, Germany) with a 63× objective oil-immersion system was used to capture biofilm images, which were then analyzed by Zen Lite 2.3 software package.

Eukaryotic cell culture and cytotoxicity assays. Immortalized human keratinocytes (HaCaT), human epidermoid carcinoma cells (A431), human dermal fibroblasts (HDF) were cultured in high-glucose Dulbecco's modified Eagle's medium (DMEM) supplemented with 10% fetal bovine serum (FBS), 1% antibiotics (Pen/strep) and 1% L-glutamine. THP-1 cells obtained from ATCC (American Type Culture Collection: TIB-202) were cultured in suspension in RPMI containing Glutamax supplemented with 10% (v/v) FBS. All cell lines were grown at 37° C. in a humidified atmosphere containing 5% CO₂. Cells seeded on 96-well plates at a density of 3×10³ cells/well 24 h prior to treatment were then incubated in the presence of increasing peptide concentrations (0-200 μmol L⁻¹) for 24, 48 and 72 hours. Following treatment with peptides, MTT assays were performed as previously described. ^{5,76,77} Briefly, cell culture supernatants were replaced with 0.5 mg/mL MTT (3-(4,5-dimethylthiazol-2-yl)-2,5-diphenyltetrazolium bromide) reagent dissolved in DMEM medium without red phenol (100 μL/well). The resulting insoluble formazan salts, after 4 h of incubation at 37° C., were then solubilized in 0.04 M HCl in anhydrous isopropanol and quantified using an automatic plate reader spectrophotometer (Synergy™ H4 Hybrid Microplate Reader, BioTek Instruments, Inc., Winooski, Vt., USA) by measuring the absorbance at wavelengths of 570 nm. Cell viability was expressed as means of the percentage values compared to control untreated cells. Lactate dehydrogenase secretion in culture medium was measured by using Lactate Dehydrogenase Activity Assay Kit (TOX7; Sigma) according to the manufacturer's instructions. At the end of cell treatment, aliquots of supernatants were collected and added to a reaction mix containing Lactate Assay Buffer, Lactate Enzyme Mix, and Lactate Substrate Mix. Absorbance at 490 nm was determined for each sample using an automatic plate reader spectrophotometer. The positive control was obtained by treating cells with lysis buffer provided by the manufacturer.

Gene expression studies. Human dermal fibroblasts were seeded into 24-well plates at a density of 1.5×10⁴ cells per well. After 24 hours, culture medium was replaced by fresh DMEM (negative control), S. aureus or A. baumannii culture at multiplicity of infection (MOI) of 0.01, LPS from E. coli O55:B5 or LTA from S. aureus (56411-57-5; Sigma) at 1μg mL⁻¹ and the samples were treated with each ApoB-derived encrypted peptide (5, 10 or 20 μmol L⁻) or with CATH-2 (2.5 or 5 μmol L⁻¹). Total RNA was extracted by using Trizol (Ambion, Carlsbad, Calif.) reagent according to the manufacturer's instructions. RNA was reverse transcribed by using the iScript cDNA synthesis kit (Bio-Rad, Veenendaal, the Netherlands) according to the manufacturer's instructions. Quantitative real-time PCR (qRT-PCR) was then performed to evaluate mRNA expression by using manufacturer's protocol (iQ SYBR Green Supermix—Bio-Rad). Reactions were performed by using the following primer sequences: for IL-8, 5′-CTGGCCGTGGCTCTCTTG' (SEQ ID NO: 2) (sense) and 5′-CCTTGGCAAAACTGCACCTT-3′ (SEQ ID NO: 3) (antisense); for IL-6, 5′-TGCAATAACCACCCCTGACC-3′ (SEQ ID NO: 4) (sense) and 5′-TGCGCAGAATGAGATGAGTTG-3′ (SEQ ID NO: 5) (antisense); and for β-actin, 5′-ATGTGGATCAGCAAGCAGGAGTA-3′ (SEQ ID NO: 6) (sense) and 5′-GCATTTGCGGTGGACGAT-3′ (SEQ ID NO: 7) (antisense). The (3-actin mRNA was used as an internal control to normalize the expression of the target genes.

Enzyme-Linked Immunosorbent Assay (ELISA). To evaluate possible pro-inflammatory effects exerted by ApoB-derived encrypted peptides, THP-1 cells, upon treatment with 100 μmol L⁻¹ phorbol 12-myristate 13-acetate (PMA) for 3 days, were plated into 96-well plates at a density of 3×10³ cells in 100 μL of medium per well. Following incubation with peptides, the medium was collected to quantify cytokines levels using human immunoassay kits (DuoSet ELISA kits, R&D Systems, Minneapolis, Minn.) according to the manufacturer's instructions. An ELISA reader set to 450 nm with a wavelength correction set to 540 nm was used to measure the optical density of each sample.

Histone deacetylase enzyme (HDAC) assay. Human keratinocytes were treated with 20 μmol L⁻¹ of each ApoB-derived peptide or with 50 nmol L⁻¹ of Tricostatin A for 30 min. Effects on Histone DeAcetylase enzyme (HDAC) activity were then evaluated by using HDAC-Glo™ I/II Assays and Screening System (Promega) according to the manufacturer's instructions.

Scarification skin infection mouse model. The anti-infective properties of ApoB-derived encrypted peptides (at working concentrations of 20; 50; 100; 150 or 200 μmol L⁻¹) were assessed in a skin murine model infected with P. aeruginosa strain PAO1 as previously described by Pane et al. 6,8,9,78 Two independent experiments were performed with 4 mice per group. Statistical significance was assessed using a one-way ANOVA

References. The following references may be relevant to the present disclosure:

(1) Santajit, S.; Indrawattana, N. Mechanisms of Antimicrobial Resistance in ESKAPE Pathogens. BioMed Research International 2016, 2016, 1-8. https://doi.org/10.1155/2016/2475067.
(2) Mulani, M. S.; Kamble, E. E.; Kumkar, S. N.; Tawre, M. S.; Pardesi, K. R. Emerging Strategies to Combat ESKAPE Pathogens in the Era of Antimicrobial Resistance: A Review. Frontiers in microbiology 2019, 10, 539. https://doi.org/10.3389/fmicb.2019.00539.
(3) Pfalzgraff, A.; Brandenburg, K.; Weindl, G. Antimicrobial Peptides and Their Therapeutic Potential for Bacterial Skin Infections and Wounds. Frontiers in Pharmacology 2018, 9. https://doi.org/10.3389/fphar.2018.00281.
(4) Pane, K.; Sgambati, V.; Zanfardino, A.; Smaldone, G.; Cafaro, V.; Angrisano, T.; Pedone, E.; Di Gaetano, S.; Capasso, D.; Haney, E. F.; Izzo, V.; Varcamonti, M.; Notomista, E.; Hancock, R. E. W.; Di Donato, A.; Pizzo, E. A New Cryptic Cationic Antimicrobial Peptide from Human Apolipoprotein E with Antibacterial Activity and Immunomodulatory Effects on Human Cells. The FEBS Journal 2016, 283 (11), 2115-2131. https://doi.org/10.1111/febs.13725.
(5) Gaglione, R.; Dell'Olmo, E.; Bosso, A.; Chino, M.; Pane, K.; Ascione, F.; Itri, F.; Caserta, S.; Amoresano, A.; Lombardi, A.; Haagsman, H. P.; Piccoli, R.; Pizzo, E.; Veldhuizen, E. J. A.; Notomista, E.; Arciello, A. Novel Human Bioactive Peptides Identified in Apolipoprotein B: Evaluation of Their Therapeutic Potential. Biochemical Pharmacology 2017, 130, 34-50. https://doi . org/10.1016/j .bcp.2017.01.009.
(6) Marcelo D. T. Tones, M. C. R. M. O. C. E. N. C. de la F.-N. Mining for Encrypted Peptide Antibiotics in the Human Proteome. Nature Biomedical Engineering 2021.
(7) Gaglione, R.; Pizzo, E.; Notomista, E.; de la Fuente-Nunez, C.; Arciello, A. Host Defence Cryptides from Human Apolipoproteins: Applications in Medicinal Chemistry. Current Topics in Medicinal Chemistry 2020, 20 (14), 1324-1337. https://doi.org/10.2174/1568026620666200427091454.
(8) Pane, K.; Cafaro, V.; Avitabile, A.; Tones, M. D. T.; Vollaro, A.; de Gregorio, E.; Catania, M. R.; di Maro, A.; Bosso, A.; Gallo, G.; Zanfardino, A.; Varcamonti, M.; Pizzo, E.; di Donato, A.; Lu, T. K.; de la Fuente-Nunez, C.; Notomista, E. Identification of Novel Cryptic Multifunctional Antimicrobial Peptides from the Human Stomach Enabled by a Computational—Experimental Platform. ACS Synthetic Biology 2018, 7 (9), 2105-2115. https://doi.org/10.102 1 / a cssynbio.8b00084.
(9) Autelitano, D. J.; Rajic, A.; Smith, A. I.; Berndt, M. C.; Ilag, L. L.; Vadas, M. The Cryptome: A Subset of the Proteome, Comprising Cryptic Peptides with Distinct Bioactivities. Drug Discovery Today. 2006. https://doi.org/10.1016/j.drudis.2006.02.003.
(10) Ueki, N.; Someya, K.; Matsuo, Y.; Wakamatsu, K.; Mukai, H. Cryptides: Functional Cryptic Peptides Hidden in Protein Structures. Biopolymers 2007, 88 (2), 190-198. https://doi.org/10.1002/bip.20687.
(11) Gaglione, R.; Dell'Olmo, E.; Bosso, A.; Chino, M.; Pane, K.; Ascione, F.; Itri, F.; Caserta, S.; Amoresano, A.; Lombardi, A.; Haagsman, H. P.; Piccoli, R.; Pizzo, E.; Veldhuizen, E. J. A.; Notomista, E.; Arciello, A. Novel Human Bioactive Peptides Identified in Apolipoprotein B: Evaluation of Their Therapeutic Potential. Biochemical pharmacology 2017, 130, 34-50. https://doi.org/10.1016/j.bcp.2017.01.009.
(12) Pane, K.; Sgambati, V.; Zanfardino, A.; Smaldone, G.; Cafaro, V.; Angrisano, T.; Pedone, E.; di Gaetano, S.; Capasso, D.; Haney, E. F.; Izzo, V.; Varcamonti, M.; Notomista, E.; Hancock, R. E. W.; di Donato, A.; Pizzo, E. A New Cryptic Cationic Antimicrobial Peptide from Human Apolipoprotein E with Antibacterial Activity and Immunomodulatory Effects on Human Cells. The FEBS Journal 2016, 283 (11), 2115-2131. https://doi.org/10.1111/febs.13725.
(13) Gaglione, R.; Cesaro, A.; Dell'Olmo, E.; della Ventura, B.; Casillo, A.; di Girolamo, R.; Velotta, R.; Notomista, E.; Veldhuizen, E. J. A.; Corsaro, M. M.; de Rosa, C.; Arciello, A. Effects of Human Antimicrobial Cryptides Identified in Apolipoprotein B Depend on Specific Features of Bacterial Strains. Scientific Reports 2019, 9 (1), 6728. https://doi.org/10.1038/s41598-019-43063-3.
(14) Shapiro, M. D.; Fazio, S. Apolipoprotein B-Containing Lipoproteins and Atherosclerotic Cardiovascular Disease. F1000Research 2017, 6, 134. https://doi.org/10.12688/f1000research.9845.1.
(15) Lister, P. D.; Wolter, D. J.; Hanson, N. D. Antibacterial-Resistant Pseudomonas Aeruginosa: Clinical Impact and Complex Regulation of Chromosomally Encoded Resistance Mechanisms. Clinical microbiology reviews 2009, 22 (4), 582-610. https://doi.org/10.1128/CMR.00040-09.
(16) Welty, F. K.; Lichtenstein, A. H.; Barrett, P. H. R.; Dolnikowski, G. G.; Schaefer, E. J. Human Apolipoprotein (Apo) B-48 and ApoB-100 Kinetics With Stable Isotopes. Arteriosclerosis, Thrombosis, and Vascular Biology 1999, 19 (12), 2966-2974. https://doi.org/10.1161/01.ATV.19.12.2966.
(17) Han, R. Plasma Lipoproteins Are Important Components of the Immune System. Microbiology and Immunology 2010, 54 (4), 246-253. https://doi.org/10.1111/j.1348-0421.2010.00203.x.
(18) Pane, K.; Durante, L.; Crescenzi, O.; Cafaro, V.; Pizzo, E.; Varcamonti, M.; Zanfardino, A.; Izzo, V.; di Donato, A.; Notomista, E. Antimicrobial Potency of Cationic Antimicrobial Peptides Can Be Predicted from Their Amino Acid Composition: Application to the Detection of “Cryptic” Antimicrobial Peptides. Journal of Theoretical Biology 2017, 419, 254-265. https://doi.org/10.1016/jitbi.2017.02.012.
(19) Gaglione, R.; Pane, K.; Dell'Olmo, E.; Cafaro, V.; Pizzo, E.; Olivieri, G.; Notomista, E.; Arciello, A. Cost-Effective Production of Recombinant Peptides in Escherichia Coli. New Biotechnology 2019, 51, 39-48. https://doi.org/10.1016/j.nbt.2019.02.004.
(20) Wiegand, I.; Hilpert, K.; Hancock, R. E. W. Agar and Broth Dilution Methods to Determine the Minimal Inhibitory Concentration (MIC) of Antimicrobial Substances. Nature protocols 2008, 3 (2), 163-175. https://doi.org/10.1038/nprot.2007.521.
(21) Rahnamaeian, M.; Vilcinskas, A. Short Antimicrobial Peptides as Cosmetic Ingredients to Deter Dermatological Pathogens. Applied Microbiology and Biotechnology 2015, 99 (21), 8847-8855. https://doi.org/10.1007/s00253-015-6926-1.
(22) Wu, X.; Hurdle, J. G. Screening for a Diamond in the Rough. Chemistry & Biology 2013, 20 (9), 1091-1092. https://doi.org/10.1016/j.chembio1.2013.09.002.
(23) te Winkel, J. D.; Gray, D. A.; Seistrup, K. H.; Hamoen, L. W.; Strahl, H. Analysis of Antimicrobial-Triggered Membrane Depolarization Using Voltage Sensitive Dyes. Frontiers in Cell and Developmental Biology 2016, 4. https://doi.org/10.3389/fce11.2016.00029.
(24) van Dijk, A.; van Eldik, M.; Veldhuizen, E. J. A.; Tjeerdsma-van Bokhoven, H. L. M.; de Zoete, M. R.; Bikker, F. J.; Haagsman, H. P. Immunomodulatory and Anti-Inflammatory Activities of Chicken Cathelicidin-2 Derived Peptides. PLOS ONE 2016, 11 (2), e0147919. https://doi.org/10.1371/journal.pone.0147919.
(25) van Dijk, A.; Molhoek, E. M.; Veldhuizen, E. J. A.; Bokhoven, J. L. M. T. T.; Wagendorp, E.; Bikker, F.; Haagsman, H. P. Identification of Chicken Cathelicidin-2 Core Elements Involved in Antibacterial and Immunomodulatory Activities. Molecular Immunology 2009, 46 (13), 2465-2473. https://doi.org/10.1016/j .molimm.2009.05.019.
(26) Boix-Lemonche, G.; Lekka, M.; Skerlavaj, B. A Rapid Fluorescence-Based Microplate Assay to Investigate the Interaction of Membrane Active Antimicrobial Peptides with Whole Gram-Positive Bacteria. Antibiotics 2020, 9 (2), 92. https://doi.org/10.3390/antibiotics9020092.
(27) Murugan, R. N.; Jacob, B.; Ahn, M.; Hwang, E.; Sohn, H.; Park, H.-N.; Lee, E.; Seo, J.-H.; Cheong, C.; Nam, K.-Y.; Hyun, J.-K.; Jeong, K.-W.; Kim, Y.; Shin, S. Y.; Bang, J. K. De Novo Design and Synthesis of Ultra-Short Peptidomimetic Antibiotics Having Dual Antimicrobial and Anti-Inflammatory Activities. PLoS ONE 2013, 8 (11), e80025. https://doi.org/10.1371/journal.pone.0080025.
(28) Gao, W.; Xing, L.; Qu, P.; Tan, T.; Yang, N.; Li, D.; Chen, H.; Feng, X. Identification of a Novel Cathelicidin Antimicrobial Peptide from Ducks and Determination of Its Functional Activity and Antibacterial Mechanism. Scientific Reports 2015, 5. https://doi.org/10.1038/srep17260.
(29) Zhang, L.; Dhillon, P.; Yan, H.; Farmer, S.; Hancock, R. E. W. Interactions of Bacterial Cationic Peptide Antibiotics with Outer and Cytoplasmic Membranes of Pseudomonas Aeruginosa. Antimicrobial Agents and Chemotherapy 2000, 44 (12). https://doi.org/10.1128/AAC.44.12.3317-3321.2000.
(30) Hou, J.; Liu, Z.; Cao, S.; Wang, H.; Jiang, C.; Hussain, M. A.; Pang, S. Broad-Spectrum Antimicrobial Activity and Low Cytotoxicity against Human Cells of a Peptide Derived from Bovine AS1-Casein. Molecules 2018, 23 (5). https://doi.org/10.3390/molecules23051220.
(31) Berditsch, M.; Jager, T.; Strempel, N.; Schwartz, T.; Overhage, J.; Ulrich, A. S. Synergistic Effect of Membrane-Active Peptides Polymyxin B and Gramicidin S on Multidrug-Resistant Strains and Biofilms of Pseudomonas Aeruginosa. Antimicrobial Agents and Chemotherapy 2015, 59 (9), 5288-5296. https://doi.org/10.1128/AAC.00682-15.
(32) Reffuveille, F.; de la Fuente-NUfiez, C.; Mansour, S.; Hancock, R. E. W. A Broad-Spectrum Antibiofilm Peptide Enhances Antibiotic Action against Bacterial Biofilms. Antimicrobial Agents and Chemotherapy 2014, 58 (9), 5363-5371. https://doi.org/10.1128/AAC.03163-14.
(33) Thappeta, K. R. v.; Vikhe, Y. S.; Yong, A. M. H.; Chan-Park, M. B.; Kline, K. A. Combined Efficacy of an Antimicrobial Cationic Peptide Polymer with Conventional Antibiotics to Combat Multidrug-Resistant Pathogens. ACS Infectious Diseases 2020, 6 (5), 1228-1237. https://doi.org/10.102 1 / a csinfecdis.0c00016.
(34) de la Fuente-NUriez, C.; Reffuveille, F.; Mansour, S. C.; Reckseidler-Zenteno, S. L.; Hernandez, D.; Brackman, G.; Coenye, T.; Hancock, R. E. W. D-Enantiomeric Peptides That Eradicate Wild-Type and Multidrug-Resistant Biofilms and Protect against Lethal Pseudomonas Aeruginosa Infections. Chemistry & Biology 2015, 22 (2), 196-205. https://doi.org/10.1016/j.chembio1.2015.01.002.
(35) Silva, O. N.; Torres, M. D. T.; Cao, J.; Alves, E. S. F.; Rodrigues, L. v.; Resende, J. M.; Lião, L. M.; Porto, W. F.; Fensterseifer, I. C. M.; Lu, T. K.; Franco, O. L.; de la Fuente-Nunez, C. Repurposing a Peptide Toxin from Wasp Venom into Antiinfectives with Dual Antimicrobial and Immunomodulatory Properties. Proceedings of the National Academy of Sciences of the United States of America 2020, 117 (43). https://doi.org/10.1073/pnas.2012379117.
(36) Wilson, H. L.; Daveson, K.; del Mar, C. B. Optimal Antimicrobial Duration for Common Bacterial Infections. Australian Prescriber 2019, 42 (1), 5. https://doi.org/10.1877 3 / austprescr.2019.001.
(37) Torres, M. D. T.; Cao, J.; Franco, 0. L.; Lu, T. K.; de la Fuente-Nunez, C. Synthetic Biology and Computer-Based Frameworks for Antimicrobial Peptide Discovery. ACS Nano 2021, 15 (2), 2143-2164. https://doi.org/10.102 1/a csnano.0c09509.
(38) Torres, M. D. T.; Sothiselvam, S.; Lu, T. K.; de la Fuente-Nunez, C. Peptide Design Principles for Antimicrobial Applications. Journal of Molecular Biology 2019, In Press. https://doi.org/10.1016/j.jmb.2018.12.015.
(39) Prada-Prada, S.; Flórez-Castillo, J.; Farfán-Garcia, A.; Guzmán, F.; Hernández-Peñaranda, I. Antimicrobial Activity of Ib-M Peptides against Escherichia Coli O157: H7. PLOS ONE 2020, 15 (2), e0229019. https://doi.org/10.1371/journal.pone.0229019.
(40) Brudzynski, K.; Sjaarda, C. Honey Glycoproteins Containing Antimicrobial Peptides, Jelleins of the Major Royal Jelly Protein 1, Are Responsible for the Cell Wall Lytic and Bactericidal Activities of Honey. PLOS ONE 2015, 10 (4), e0120238. https://doi.org/10.1371/journal.pone.0120238.
(41) el Shazely, B.; Yu, G.; Johnston, P. R.; Rolff, J. Resistance Evolution Against Antimicrobial Peptides in Staphylococcus Aureus Alters Pharmacodynamics Beyond the MIC. Frontiers in Microbiology 2020, 11. https://doi.org/10.3389/fmicb.2020.00103.
(42) Crouzet, M.; le Senechal, C.; Brozel, V. S.; Costaglioli, P.; Barthe, C.; Bonneu, M.; Garbay, B.; Vilain, S. Exploring Early Steps in Biofilm Formation: Set-up of an Experimental System for Molecular Studies. BMC Microbiology 2014, 14 (1), 253. https://doi.org/10.1186/s12866-014-0253-z.
(43) de La Fuente-Núñiez, C.; Korolik, V.; Bains, M.; Nguyen, U.; Breidenstein, E. B. M.; Horsman, S.; Lewenza, S.; Burrows, L.; Hancock, R. E. W. Inhibition of Bacterial Biofilm Formation and Swarming Motility by a Small Synthetic Cationic Peptide. Antimicrobial Agents and Chemotherapy 2012, 56 (5). https://doi.org/10.1128/AAC.00064-12.
(44) Perry, J. A.; Wright, G. D. Forces Shaping the Antibiotic Resistome. BioEssays 2014, 36 (12), 1179-1184. https://doi.org/10.1002/bies.201400128.
(45) Fujita, Y.; Taguchi, H. Current Status of Multiple Antigen-Presenting Peptide Vaccine Systems: Application of Organic and Inorganic Nanoparticles. Chemistry Central Journal 2011, 5 (1), 48. https://doi.org/10.1186/1752-153X-5-48.
(46) Veas, F.; Dubois, G. IL-22 Induces an Acute-Phase Response Associated to a Cohort of Acute Phase Proteins and Antimicrobial Peptides as Players of Homeostasis. In Acute Phase Proteins—Regulation and Functions of Acute Phase Proteins; InTech, 2011. https://doi.org/10.5772/20386.
(47) Wang, C.; Tian, L.-L.; Li, S.; Li, H.-B.; Zhou, Y.; Wang, H.; Yang, Q.-Z.; Ma, L.-J.; Shang, D.-J. Rapid Cytotoxicity of Antimicrobial Peptide Tempoprin-1CEa in Breast Cancer Cells through Membrane Destruction and Intracellular Calcium Mechanism. PLoS ONE 2013, 8 (4), e60462. https://doi.org/10.1371/journal.pone.0060462.
(48) Tornesello, A. L.; Borrelli, A.; Buonaguro, L.; Buonaguro, F. M.; Tornesello, M. L. Antimicrobial Peptides as Anticancer Agents: Functional Properties and Biological Activities. Molecules. 2020. https://doi.org/10.3390/molecules25122850.
(49) Pedron, C. N.; de Oliveira, C. S.; da Silva, A. F.; Andrade, G. P.; da Silva Pinhal, M. A.; Cerchiaro, G.; da Silva Junior, P. I.; da Silva, F. D.; Torres, M. D. T.; Oliveira, V. X. The Effect of Lysine Substitutions in the Biological Activities of the Scorpion Venom Peptide VmCT1. European Journal of Pharmaceutical Sciences 2019, 136. https://doi.org/10.1016/j.ejps.2019.06.006.
(50) Lu, Y.; Zhang, T.-F.; Shi, Y.; Zhou, H.-W.; Chen, Q.; Wei, B.-Y.; Wang, X.; Yang, T.-X.; Chinn, Y. E.; Kang, J.; Fu, C.-Y. PFR Peptide, One of the Antimicrobial Peptides Identified from the Derivatives of Lactoferrin, Induces Necrosis in Leukemia Cells. Scientific Reports 2016, 6 (1), 20823. https://doi.org/10.1038/srep20823.
(51) Méndez-Samperio, P. Recent Advances in the Field of Antimicrobial Peptides in Inflammatory Diseases. Advanced Biomedical Research 2013, 2 (1), 50. https://doi.org/10.4103/2277-9175.114192.
(52) Haisma, E. M.; Rietveld, M. H.; de Breij, A.; van Dissel, J. T.; el Ghalbzouri, A.; Nibbering, P. H. Inflammatory and Antimicrobial Responses to Methicillin-Resistant Staphylococcus Aureus in an in Vitro Wound Infection Model. PLoS ONE 2013, 8 (12), e82800. https://doi.org/10.1371/journal.pone.0082800.
(53) Totte, J. E. E.; van der Feltz, W. T.; Bode, L. G. M.; van Belkum, A.; van Zuuren, E. J.; Pasmans, S. G. M. A. A Systematic Review and Meta-Analysis on Staphylococcus Aureus Carriage in Psoriasis, Acne and Rosacea. European Journal of Clinical Microbiology & Infectious Diseases 2016, 35 (7), 1069-1077. https://doi.org/10.1007/s10096-016-2647-3.
(54) Hoffmann, J. P.; Friedman, J. K.; Wang, Y.; McLachlan, J. B.; Sammarco, M. C.; Morici, L. A.; Roy, C. J. In Situ Treatment With Novel Microbiocide Inhibits Methicillin Resistant Staphylococcus Aureus in a Murine Wound Infection Model. Frontiers in Microbiology 2020, 10. https://doi.org/10.3389/fmicb.2019.03106.
(55) Pietrzak, A.; Chabros, P.; Grywalska, E.; Kicifiski, P.; Pietrzak-Franciszkiewicz, K.; Krasowska, D.; Kandzierski, G. Serum Lipid Metabolism in Psoriasis and Psoriatic Arthritis—An Update. Archives of medical science:AMS 2019, 15 (2), 369-375. https://doi.org/10.5114/aoms.2018.74021.
(56) Hall, P. R.; Elmore, B. O.; Spang, C. H.; Alexander, S. M.; Manifold-Wheeler, B. C.; Castleman, M. J.; Daly, S. M.; Peterson, M. M.; Sully, E. K.; Femling, J. K.; Otto, M.; Horswill, A. R.; Timmins, G. S.; Gresham, H. D. Nox2 Modification of LDL Is Essential for Optimal Apolipoprotein B-Mediated Control of Agr Type III Staphylococcus Aureus Quorum-Sensing. PLoS Pathogens 2013, 9 (2), e1003166. https://doi.org/10.1371/journal.ppat.1003166.
(57) Pernot, M.; Vanderesse, R.; Frochot, C.; Guillemin, F.; Barberi-Heyob, M. Stability of Peptides and Therapeutic Success in Cancer. Expert Opinion on Drug Metabolism & Toxicology 2011, 7 (7), 793-802. https://doi.org/10.1517/17425255.2011.574126.
(58) Starr, C. G.; Wimley, W. C. Antimicrobial Peptides Are Degraded by the Cytosolic Proteases of Human Erythrocytes. Biochimica et Biophysica Acta (BBA)—Biomembranes 2017, 1859 (12), 2319-2326. https://doi.org/10.1016/j.bbamem.2017.09.008.
(59) Böttger, R.; Hoffmann, R.; Knappe, D. Differential Stability of Therapeutic Peptides with Different Proteolytic Cleavage Sites in Blood, Plasma and Serum. PLOS ONE 2017, 12 (6), e0178943. https://doi.org/10.1371/journal.pone.0178943.
(60) Garton, M.; Nim, S.; Stone, T. A.; Wang, K. E.; Deber, C. M.; Kim, P. M. Method to Generate Highly Stable D-Amino Acid Analogs of Bioactive Helical Peptides Using a Mirror Image of the Entire PDB. Proceedings of the National Academy of Sciences 2018, 115 (7), 1505-1510. https://doi.org/10.1073/pnas.1711837115.
(61) Cardoso, M. H.; Cândido, E. S.; Oshiro, K. G. N.; Rezende, S. B.; Franco, O.L. Peptides Containing d -Amino Acids and Retro-Inverso Peptides. In Peptide Applications in Biomedicine, Biotechnology and Bioengineering; Elsevier, 2018; pp 131-155. https://doi.org/10.1016/B978-0-08-100736-5.00005-3.
(62) Oliva, R.; Chino, M.; Pane, K.; Pistorio, V.; de Santis, A.; Pizzo, E.; D′Errico, G.; Pavone, V.; Lombardi, A.; del Vecchio, P.; Notomista, E.; Nastri, F.; Petraccone, L. Exploring the Role of Unnatural Amino Acids in Antimicrobial Peptides. Scientific Reports 2018, 8 (1), 8888. https://doi.org/10.1038/s41598-018-27231-5.
(63) Torres, M. D. T.; Pedron, C. N.; Higashikuni, Y.; Kramer, R. M.; Cardoso, M. H.; Oshiro, K. G. N.; Franco, O. L.; Silva Junior, P. I.; Silva, F. D.; Oliveira Junior, V. X.; Lu, T. K.; de la Fuente-Nunez, C. Structure-Function-Guided Exploration of the Antimicrobial Peptide Polybia-CP Identifies Activity Determinants and Generates Synthetic Therapeutic Candidates. Communications Biology 2018, 1 (1), 221. https://doi.org/10.1038/s42003-018-0224-2.
(64) Pedron, C. N.; Torres, M. D. T.; Lima, J. A. D. S.; Silva, P. I.; Silva, F. D.; Oliveira, V. X. Novel Designed VmCT1 Analogs with Increased Antimicrobial Activity. European Journal of Medicinal Chemistry 2017, 126. https://doi.org/10.1016/j.ejmech.2016.11.040.
(65) Torres, M. D. T.; Pedron, C. N.; da Silva Lima, J. A.; da Silva, P. I.; da Silva, F. D.; Oliveira, V. X. Antimicrobial Activity of Leucine-Substituted Decoralin Analogs with Lower Hemolytic Activity. Journal of Peptide Science 2017, 23, 818-823. https://doi.org/10.1002/psc.3029.
(66) Torres, M. D. T.; Pedron, C. N.; Araújo, I.; Silva, P. I.; Silva, F. D.; Oliveira, V. X. Decoralin Analogs with Increased Resistance to Degradation and Lower Hemolytic Activity. ChemistrySelect 2017, 2 (1), 18-23. https://doi.org/10.1002/slct.201601590.
(67) Grishin, D. v.; Zhdanov, D. D.; Pokrovskaya, M. v.; Sokolov, N. N. D-Amino Acids in Nature, Agriculture and Biomedicine. All Life 2020, 13 (1), 11-22. https://doi.org/10.1080/21553769.2019.1622596.
(68) Dryden, M. S. Complicated Skin and Soft Tissue Infection. The Journal of antimicrobial chemotherapy 2010, 65 Suppl 3 (Supplement 3), iii35-44. https://doi.org/10.1093/jac/dkq302.
(69) Becerra, S. C.; Roy, D. C.; Sanchez, C. J.; Christy, R. J.; Burmeister, D. M. An Optimized Staining Technique for the Detection of Gram Positive and Gram Negative Bacteria within Tissue. BMC Research Notes 2016, 9 (1). https://doi.org/10.1186/s13104-016-1902-0.
(70) Serra, R.; Grande, R.; Butrico, L.; Rossi, A.; Settimio, U. F.; Caroleo, B.; Amato, B.; Gallelli, L.; de Franciscis, S. Chronic Wound Infections: The Role of Pseudomonas Aeruginosa and Staphylococcus Aureus. Expert Review of Anti-infective Therapy 2015, 13 (5), 605-613. https://doi.org/10.1586/14787210.2015.1023291.
(71) Gaglione, R.; Smaldone, G.; Cesaro, A.; Rumolo, M.; de Luca, M.; di Girolamo, R.; Petraccone, L.; del Vecchio, P.; Oliva, R.; Notomista, E.; Pedone, E.; Arciello, A. Impact of a Single Point Mutation on the Antimicrobial and Fibrillogenic Properties of Cryptides from Human Apolipoprotein B. Pharmaceuticals 2021, 14 (7). https://doi.org/10.3390/ph14070631.
(72) Powell, M. F.; Stewart, T.; Otvos, L.; Urge, L.; Gaeta, F. C. A.; Sette, A.; Arrhenius, T.; Thomson, D.; Soda, K.; Colon, S. M. Peptide Stability in Drug Development. II. Effect of Single Amino Acid Substitution and Glycosylation on Peptide Reactivity in Human Serum. Pharmaceutical research 1993, 10 (9), 1268-1273. https://doi.org/10.1023/a:1018953309913.
(73) Torres, M. D. T.; Pedron, C. N.; Araújo, I.; Silva, P. I.; Silva, F. D.; Oliveira, V. X. Decoralin Analogs with Increased Resistance to Degradation and Lower Hemolytic Activity. ChemistrySelect 2017, 2 (1). https://doi.org/10.1002/slct.201601590.
(74) Gaglione, R.; Cesaro, A.; Dell'Olmo, E.; di Girolamo, R.; Tartaglione, L.; Pizzo, E.; Arciello, A. Cryptides Identified in Human Apolipoprotein B as New Weapons to Fight Antibiotic Resistance in Cystic Fibrosis Disease. International Journal of Molecular Sciences 2020, 21 (6), 2049. https://doi.org/10.3390/ijms21062049.
(75) Dell'Olmo, E.; Gaglione, R.; Sabbah, M.; Schibeci, M.; Cesaro, A.; di Girolamo, R.; Porta, R.; Arciello, A. Host Defense Peptides Identified in Human Apolipoprotein B as Novel Food Biopreservatives and Active Coating Components. Food Microbiology 2021. https://doi.org/10.1016/j.fm.2021.103804.
(76) Gaglione, R.; Pirone, L.; Farina, B.; Fusco, S.; Smaldone, G.; Aulitto, M.; Dell'Olmo, E.; Roscetto, E.; del Gatto, A.; Fattorusso, R.; Notomista, E.; Zaccaro, L.; Arciello, A.; Pedone, E.; Contursi, P. Insights into the Anticancer Properties of the First Antimicrobial Peptide from Archaea. Biochimica et Biophysica Acta (BBA)—General Subjects 2017, 1861 (9), 2155-2164. https://doi.org/10.1016/j.bbagen.2017.06.009.
(77) Gaglione, R.; Cesaro, A.; Dell'olmo, E.; di Girolamo, R.; Tartaglione, L.; Pizzo, E.; Arciello, A. Cryptides Identified in Human Apolipoprotein b as New Weapons to Fight Antibiotic Resistance in Cystic Fibrosis Disease. International Journal of Molecular Sciences 2020, 21 (6). https://doi.org/10.3390/ijms21062049.
(78) Pfalzgraff, A.; Brandenburg, K.; Weindl, G. Antimicrobial Peptides and Their Therapeutic Potential for Bacterial Skin Infections and Wounds. Frontiers in Pharmacology 2018, 9. https://doi.org/10.3389/fphar.2018.00281.

What is claimed:

1. A peptide, wherein said peptide is SEQ ID NO:1.

2. A peptide having an amino acid sequence that is at least 90% homologous to SEQ ID NO:1.

3. A peptide having an amino acid sequence that is at least 95% homologous to SEQ ID NO:1.

4. A peptide having an amino acid sequence that varies from SEQ ID NO:1 by one, two, or three individual amino acids.

5. A composition comprising a peptide according to claim 1 and a pharmaceutically acceptable carrier or excipient.

6. A method of treating a microbial infection comprising administering to a subject in need thereof a pharmaceutically effective amount of a peptide of claim 1.

7. The method according to claim 6, wherein the microbial infection is includes Gram-negative bacteria.

8. The method according to claim 6, further comprising administering to the subject an additional antimicrobial agent.

9. The method according to claim 8, wherein the additional antimicrobial agent is an antibiotic.

10. The method according to claim 8, wherein the additional antimicrobial agent is active against bacterial membranes.

11. The method according to claim 8, wherein the additional antimicrobial agent is an inhibitor of bacterial protein synthesis.

12. The method according to claim 8, wherein the additional antimicrobial agent is active against bacterial cell walls.

13. A method for treating inflammation comprising administering to a subject in need thereof a pharmaceutically effective amount of a peptide of a peptide of claim 1.

14. The method according to claim 13, further comprising administering to the subject an additional antimicrobial agent.

15. The method according to claim 14, wherein the additional antimicrobial agent is an antibiotic.

16. The method according to claim 14, wherein the additional antimicrobial agent is active against bacterial membranes.

17. The method according to claim 14, wherein the additional antimicrobial agent is an inhibitor of bacterial protein synthesis.

18. The method according to claim 14, wherein the additional antimicrobial agent is active against bacterial cell walls.

19. A method comprising contacting a biofilm with an effective amount of a peptide of claim 1.

20. The method according to claim 19, wherein the biofilm comprises Gram-negative bacteria.

21. A method for reducing biofilm formation on a surface comprising contacting the surface with an effective amount of a peptide of claim 1.

22. The method according to claim 21, wherein the biofilm comprises Gram-negative bacteria.
